SEQUENCE LISTING (43 sequences)

SEQ ID NO: 1 - DNA, 19 bp, HIV-1
  misc_feature (1)..(19): 143 to 161 5′ LTR U3
gctagtaccagttgagcca

SEQ ID NO: 2 - DNA, 19 bp, HIV-1
  misc_feature (1)..(19): strain BaL, subtypes B, D, F, H, J, K (143 to 161 5′ LTR U3)
gctagtaccagttgagcca

SEQ ID NO: 3 - DNA, 19 bp, HIV-1
  misc_feature (1)..(19): subtype A (143 to 161 5′ LTR U3)
gctagtaccagttgatcca

SEQ ID NO: 4 - DNA, 19 bp, HIV-1
  misc_feature (1)..(19): subtype C (143 to 161 5′ LTR U3)
gctagtgccagttgaccca

SEQ ID NO: 5 - DNA, 19 bp, HIV-1
  misc_feature (1)..(19): subtype G (143 to 161 5′ LTR U3)
gctagtaccagtggaccca

SEQ ID NO: 6 - RNA, 19 nt, Artificial Sequence
  Synthetic RNA duplex sense strand (143 to 161 5′ LTR U3)
gcuaguaccaguugagcca

SEQ ID NO: 7 - RNA, 19 nt, Artificial Sequence
  Synthetic RNA duplex antisense strand (143 to 161 5′ LTR U3)
uggcucaacugguacuagc

SEQ ID NO: 8 - RNA, 57 nt, Artificial Sequence
  Synthetic shRNA (143 to 161 5′ LTR U3)
gcuaguaccaguugagccacugugaagccacagauggguggcucaacugguacuagc

SEQ ID NO: 9 - DNA, 19 bp, HIV-1
  misc_feature (1)..(19): 136 to 154 5′ LTR U3
gctacaagctagtaccagt

SEQ ID NO: 10 - DNA, 19 bp, HIV-1
  misc_feature (1)..(19): strain SF162, subtypes A, B, F, H, J, K (136 to 154 5′ LTR U3)
gcttcaagctagtaccagt

SEQ ID NO: 11 - DNA, 19 bp, HIV-1
  misc_feature (1)..(19): subtype C (136 to 154 5′ LTR U3)
gcttcaagctagtgccagt

SEQ ID NO: 12 - DNA, 19 bp, HIV-1
  misc_feature (1)..(19): subtype D (136 to 154 5′ LTR U3)
gcttcgagctagtaccagt

SEQ ID NO: 13 - DNA, 19 bp, HIV-1
  misc_feature (1)..(19): subtype G (136 to 154 5′ LTR U3)
gcttcaaactagtaccaat

SEQ ID NO: 14 - RNA, 19 nt, Artificial Sequence
  Synthetic RNA duplex sense strand (136 to 154 5′ LTR U3)
gcuacaagcuaguaccagu

SEQ ID NO: 15 - RNA, 19 nt, Artificial Sequence
  Synthetic RNA duplex antisense strand (136 to 154 5′ LTR U3)
acugguacuagcuuguagc

SEQ ID NO: 16 - RNA, 57 nt, Artificial Sequence
  Synthetic shRNA (136 to 154 5′ LTR U3)
gcuucaagcuaguaccagucugugaagccacagaugggacugguacuagcuugaagc

SEQ ID NO: 17 - DNA, 19 bp, HIV-1
  misc_feature (1)..(19): 205 to 223 5′ LTR U3
acaccctatgagcctgcat

SEQ ID NO: 18 - DNA, 19 bp, HIV-1
  misc_feature (1)..(19): strain SF162, subtypes A, B, H, J, K (205 to 223 5′ LTR U3)
acaccctatgagcctgcat

SEQ ID NO: 19 - DNA, 19 bp, HIV-1
  misc_feature (1)..(19): subtype C and D (205 to 223 5′ LTR U3)
acaccctatgagccagcat

SEQ ID NO: 20 - DNA, 19 bp, HIV-1
  misc_feature (1)..(19): Subtype F (205 to 223 5′ LTR U3)
acaccccatgagccaacat

SEQ ID NO: 21 - DNA, 19 bp, HIV-1
  misc_feature (1)..(19): Subtype G (205 to 223 5′ LTR U3)
acaccccatctgccagcat

SEQ ID NO: 22 - RNA, 19 nt, Artificial Sequence
  Synthetic RNA duplex sense strand (205 to 223 5′ LTR U3)
acacccugugagccugcau

SEQ ID NO: 23 - RNA, 19 nt, Artificial Sequence
  Synthetic RNA duplex antisense strand (205 to 223 5′ LTR U3)
augcaggcucacagggugu

SEQ ID NO: 24 - RNA, 57 nt, Artificial Sequence
  Synthetic shRNA (205 to 223 5′ LTR U3)
acacccuaugagccugcaucugugaagccacagaugggaugcaggcucauagggugu

SEQ ID NO: 25 - RNA, 51 nt, Unknown
  shRNA targeting CCR5 receptor
gagcaagcucaguuuacaccuuguccgacgguguaaacugagcuugcucuu

SEQ ID NO: 26 - protein, 135 aa, Artificial Sequence [sequence omitted]

SEQ ID NO: 27 - DNA, 408 bp, Artificial Sequence
  Synthetic C46 HIV fusion inhibitor
atgggagcaggagcaaccggaagggcaatggacggaccacggctgctgctgctgctgctgctgggcgtgagcctgggcggcgcccggagctggatggagtgggaccgggagatcaacaactacaccagcctgatccacagcctgatcgaggagagccagaaccagcaggagaagaacgagcaggagctgctggagctggacaagtgggccagcctgtggaactggttccggagcgagcggaagtgctgcgtggagtgcccaccatgcccagcaccaccagtggcaggacccctgatcgcactggtgaccagcggagccctgctggccgtgctgggcatcacaggctacttcctgatgaaccggcggagctggagccccaccggcgagcggctggagctggagccctga

SEQ ID NO: 28 - protein, 493 aa, Homo sapiens
  misc_feature (1)..(493): human TRIM5-alpha protein
MASGILVNVKEEVTCPICLELLTQPLSLDCGHSFCQACLTANHKKSMLDKGESSCPVCRISYQPENIRPNRHVANIVEGLREVKLSPEGQKVDHCARHGEKLLLFCQEDGKVICWLCERSQEHRGHHTFLTEEVAREYQVKLQAALEMLRQKQQEAEELEADIREEKASWKTQIQYDKTNVLADFEQLRDILDWEESNELQNLEKEEEDILKSLTNSETEMVQQTQSLRELISDLEHRLQGSVMELLQGVDGVIKRTENVTLKKPETFPKNQRRVFRAPDLKGMLEVFRELTDVRRYWVDVTVAPNNISCAVISEDKRQVSSPKPQIIYGARGTRYQTFVNFNYCTGILGSQSITSGKSYWEVDVSKKTAWILGVCAGFQPDAMCNIEKNENYQPNYGYWVIGLEEGVKCSAFQDSSFHTPSVPFIVPLSVLICPDRVGVFLDYEACTVSFFNITNHGFLIYKFSHCSFSQPVFPYLNPRKCGVPMTLCSPSS

SEQ ID NO: 29 - DNA, 1482 bp, Homo sapiens
  misc_feature (1)..(1482): human TRIM5-alpha protein nucleic acid sequence
atggcttctggaatcctggttaatgtaaaggaggaggtgacctgccccatctgcctggaactcctgacacaacccctgagcctggactgcggccacagcttctgccaagcatgcctcactgcaaaccacaagaagtccatgctagacaaaggagagagtagctgccctgtgtgccggatcagttaccagcctgagaacatacggcctaatcggcatgtagccaacatagtggagaagctcagggaggtcaagttgagcccagaggggcagaaagttgatcattgtgcacgccatggagagaaacttctactcttctgtcaggaggacgggaaggtcatttgctggctttgtgagcggtctcaggagcaccgtggtcaccacacgttcctcacagaggaggttgcccgggagtaccaagtgaagctccaggcagctctggagatgctgaggcagaagcagcaggaagctgaagagttagaagctgacatcagagaagagaaagcttcctggaagactcaaatacagtatgacaaaaccaacgtcttggcagattttgagcaactgagagacatcctggactgggaggagagcaatgagctgcaaaacctggagaaggaggaggaagacattctgaaaagccttacgaactctgaaactgagatggtgcagcagacccagtccctgagagagctcatctcagatctggagcatcggctgcaggggtcagtgatggagctgcttcagggtgtggatggcgtcataaaaaggacggagaacgtgaccttgaagaagccagaaacttttccaaaaaatcaaaggagagtgtttcgagctcctgatctgaaaggaatgctagaagtgtttagagagctgacagatgtccgacgctactgggttgatgtgacagtggctccaaacaacatttcatgtgctgtcatttctgaagataagagacaagtgagctctccgaaaccacagataatatatggggcacgagggacaagataccagacatttgtgaatttcaattattgtactggcatcctgggctctcaaagtatcacatcagggaaacattactgggaggtagacgtgtccaagaaaactgcttggatcctgggggtatgtgctggcttccaacctgatgcaatgtgtaatattgaaaaaaatgaaaattatcaacctaaatacggctactgggttatagggttagaggaaggagttaaatgtagtgctttccaggatagttccttccatactccttctgttcctttcattgtgcccctctctgtgattatttgtcctgatcgtgttggagttttcctagactatgaggcttgcactgtctcattcttcaatatcacaaaccatggatttctcatctataagttttctcagtgttctttttctcagcctgtatttccatatttaaatcctagaaaatgtggagtccccatgactctgtgctcaccaagctcttga

SEQ ID NO: 30 - DNA, 30 bp, Artificial Sequence
  Synthetic HIV-gag sense primer
agtggggggacatcaagcagccatgcaaat

SEQ ID NO: 31 - DNA, 28 bp, Artificial Sequence
  Synthetic HIV-gag antisense primer
tactagtagttcctgctatgtcacttcc

SEQ ID NO: 32 - DNA, 29 bp, Artificial Sequence
  Synthetic HIV-gag detection probe
  misc_feature: 5′ FAM / 3′ TAMRA
atcaatgaggaagctgcagaatgggatag

SEQ ID NO: 33 - DNA, 25 bp, Artificial Sequence
  Synthetic Beta-actin sense primer
tcacccacactgtgcccatctacga

SEQ ID NO: 34 - DNA, 25 bp, Artificial Sequence
  Synthetic Beta-actin anti-sense primer
cagcggaaccgctcattgccaatgg

SEQ ID NO: 35 - DNA, 25 bp, Artificial Sequence
  Synthetic Beta-actin detection probe
  misc_feature: 5′ FAM / 3′ TAMRA
atgccctcccccatgccatcctgcg

SEQ ID NO: 36 - DNA, 19 bp, HIV-1
  misc_feature (1)..(19): 350 to 368 5′ LTR U3-PromA
gggactttccgctggggac

SEQ ID NO: 37 - DNA, 19 bp, HIV-1
  misc_feature (1)..(19): Strain NL4.3, subtypes A, B, D, F, H, J, K (350 to 368 5LTR U3-PromA)
gggactttccgctggggac

SEQ ID NO: 38 - DNA, 18 bp, HIV-1
  misc_feature (1)..(18): Subtype C (350 to 368 5LTR U3-PromA)
gggactttccactggggc

SEQ ID NO: 39 - DNA, 19 bp, HIV-1
  misc_feature (1)..(19): Subtype G (350 to 368 5LTR U3-PromA)
gggactttccgcctgggac

SEQ ID NO: 40 - RNA, 19 nt, Artificial Sequence
  Synthetic RNA duplex sense strand (350 to 368 5 LTR U3-PromA)
gggacuuuccgcuggggac

SEQ ID NO: 41 - RNA, 19 nt, Artificial Sequence
  Synthetic RNA duplex antisense strand (350 to 368 5 LTR U3-PromA)
guccccagcggaaaguccc

SEQ ID NO: 42 - RNA, 57 nt, Artificial Sequence
  Synthetic shRNA (350 to 368 5LTR U3-PromA)
gggacuuuccgcuggggaccugugaagccacagaugggguccccagcggaaaguccc

SEQ ID NO: 43 - RNA, 19 nt, Artificial Sequence
  Synthetic shRNA Loop sequence
cugugaagccacagauggg

(19) United States
(12) Patent Application Publication
(10) Pub. No.: US 2018/0112220 A1
(43) Pub. Date: Apr. 26, 2018

(54) GENE THERAPEUTIC FOR THE TREATMENT OF HIV AND USES THEREOF

(71) Applicants: Calimmune, Inc., Tucson, AZ (US); Calimmune Australia PTY LTD, Darlinghurst (AU); St. Vincent's Hospital Sydney Limited, Darlinghurst (AU); Newsouth Innovations PTY Limited, Sydney (AU)

(72) Inventors: Anthony Dominic Kelleher, Bangor (AU); Kazuo Suzuki, Sydney (AU); Chantelle Lisa Evelyn Ahlenstiel, Ryde (AU); Geoffrey Phillip Symonds, Rose Bay (AU)

(21) Appl. No.: 15/810,222

(22) Filed: Nov. 13, 2017

Related U.S. Application Data
(63) Continuation of application No. PCT/US2016/017931, filed on Feb. 15, 2016.
(60) Provisional application No. 62/163,332, filed on May 18, 2015.

(51) Int. Cl. C12N 15/113; C12N 15/86; A61K 35/28

(52) U.S. Cl. CPC C12N 15/1132; C12N 15/86; A61K 35/28; A61K 48/00; C12N 2310/14; C12N 2310/531; C12N 2740/16043; C12N 15/1138

(57) ABSTRACT

The present disclosure provides for an expression vector comprising (i) at least one nucleic acid sequence encoding a transcriptional gene silencing element; and (ii) at least two other nucleic acid sequences selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry. The combination of the three mechanisms in a single expression vector provides for novel means of inhibiting HIV-I.

### CROSS-REFERENCE TO RELATED APPLICATIONS

This application is a continuation of International Application No. PCT/US2016/017931, filed Feb. 15, 2016; which application claims the benefit of the filing date of U.S. Provisional Patent Application No. 62/163,332 filed May 18, 2015, the disclosure of which is hereby incorporated by reference herein in its entirety.

### FIELD OF DISCLSOURE

This disclosure generally relates to the fields of molecular biology and virology. In particular, the disclosure relates to an expression vector useful in the treatment and prevention of HIV infections as well as a method of inhibiting HIV infection and replication in tissue culture systems, in animal model systems and in HIV-1 infected subjects.

### BACKGROUND OF THE DISCLSOURE

HIV-1 is the causative agent of Acquired Immunodeficiency Syndrome (AIDS) with of the order of 30 million individuals infected world-wide. HIV causes the immune system to fail and increases the probability of death due to opportunistic infections. HIV infection is a major global health problem as evidenced by its designation as a pandemic by the World Health Organization. Most people who are infected with HIV, particularly in the developing world, eventually develop AIDS, which claims the lives of more than one million people every year.

HIV-1 belongs to the retroviridae family of viruses, and is an enveloped virus whose genome consists of two single stranded RNA molecules (ssRNA). The primary target of HIV-1 is CD4+ expressing cells, such as CD4+ T cells. A glycoprotein of the HIV-1 virus interacts with the CD4 molecule of target cells and with chemokine co-receptors, CCRS or CXCR4 on the surface of target cells. Following fusion and entry into the target cell, the nucleocapsid containing the viral genome dissociates, releasing the contents of the virus, including the ssRNA, into the cytoplasm. A reverse transcriptase (RT) enzyme of HIV-1 synthesizes viral double stranded DNA (dsDNA) from the ssRNA genome. Following synthesis of the double stranded HIV-1 DNA molecule, the HIV-1 DNA is integrated into the host genome.

The integrated HIV-1 DNA is flanked by identical 5′ and 3′ long terminal repeat sequences (LTR) from which HIV-1 can initiate transcription of the integrated HIV-1 genome. Transcription of the viral DNA requires transcription factors, such as NF-kB, which are upregulated in activated T cells. As a consequence, viral transcription is most active in the T cell following activation of the T cell, such as during infection. Viral RNA resulting from transcription of the integrated HIV-1 genome is subsequently translated and packaged into virus particles which then exit the cell to become infectious virus.

Therapy for HIV-1 infection includes combination antiretroviral therapy (cART). cART, which includes combinations of nucleoside analogue reverse transcriptase inhibitors, protease inhibitors, non-nucleoside reverse transcriptase inhibitors, integrase and fusion inhibitors, slows HIV progression. This, in turn, dramatically decreases the morbidity and mortality rate from HIV/AIDS in regions of the world where the therapy is available. However, cART does not cure or completely eliminate all the symptoms of HIV/AIDS. Also, cART therapy can be compromised by drug resistant mutations, and has a range of side effects which can be serious and which appear to be cumulative. Further, interruption of cART therapy almost invariably leads to the re-emergence of detectable viral replication and the progression to AIDS and has been shown to be associated with an increased incidence of all causes of mortality and serious non-AIDS events. For these reasons, as well as the high cost of cART and need for strict adherence, such therapy can be relatively ineffective for a large number of patients.

HIV-based lentiviral vectors are rapidly becoming the retrovirus vector system of choice for research and clinical gene transfer applications. The enhanced ability of lentiviral vectors to transduce both quiescent stem cells and non-dividing terminally differentiated cells has led to the development of a wide range of therapeutic gene delivery vectors, as well as promising research tools, such as short hairpin RNA (shRNA) gene knockdown libraries and vectors for induction of pluripotency in terminally differentiated cells. Early gamma-retroviral clinical gene therapy vectors restored immune function in patients with X-linked severe combined immunodeficiency (SCID-X1), but they were subsequently found to cause proliferative disorders via transactivation of proto-oncogenes. Newer lentiviral vector designs may significantly reduce that risk, and they await clinical testing for final validation of their predicted safety. The field remains in flux and the outcomes of the clinical testing are unpredictable.

For instance, Trobridge et al., PLoS One, Vol. 4: e7693, 2009 (hereinafter “Trobridge”) teach that insertion of a pol III-driven shRNA in the same expression vector as a C46 fusion inhibitor gene driven by a separate promoter resulted in a significant reduction in C46 expression as compared to a vector that did not include the shRNA (see page 5, left column, first paragraph and FIG. 3 of Trobridge). Consequently, the dual vector was substantially less efficacious (more than 27-fold less effective) against HIV infection than the vector encoding the C46 fusion inhibitor alone (see Id.). In addition, the dual vector exhibited significantly lower rates of anti-HIV gene transfer to hematopoietic cells as compared to the vector encoding the C46 fusion inhibitor alone (see pages 6 and 7, Table 1, and FIG. 6). Trobridge attributed the poor expression of C46 to the inclusion of the shRNA-encoding sequence in the vector (see page 7, right column, first paragraph and page 9, left column, third paragraph). Trobridge also discloses that low gene transfer levels are a major roadblock in clinical trials for anti-HIV gene therapy (see, e.g., abstract, page 2, left column, first full paragraph, and page 8, right column, second full paragraph).

### SUMMARY OF THE DISCLSOURE

The present disclosure provides a novel therapeutic approach for treating, preventing and/or reducing HIV infection in which a combination of different steps in viral infection are targeted by gene therapy. For instance, the present disclosure provides a vector encoding an inhibitor of viral entry into a host cell, an inhibitor of viral fusion and/or an inhibitor of viral replication and an inhibitor of HIV-1 gene transcription.

In some aspects of the present disclosure are expression vectors. Thus, in accordance with one aspect of the present disclosure is an expression vector comprising (i) at least one nucleic acid sequence encoding a transcriptional gene silencing element; and (ii) at least two other nucleic acid sequences selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry. In some embodiments, the at least one nucleic acid sequence encoding a transcriptional gene silencing element is a silencing nucleic acid which targets a sequence of the 5′ LTR of HIV. In some embodiments, the at least one nucleic acid sequence encoding a transcriptional gene silencing element is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1 (the sequence from position 350 to 368 is also referred to herein as “PromA”). In some embodiments, the at least two other nucleic acid sequences comprise a nucleic acid sequence that encodes a nucleic acid molecule that inhibits an HIV co-receptor; and a nucleic acid sequence that encodes an HIV fusion inhibitor protein. In other embodiments, the at least two other nucleic acid sequences comprise a nucleic acid sequence that encodes a nucleic acid molecule that inhibits an HIV co-receptor; and a nucleic acid sequence encoding an inhibitor of HIV replication.

In some embodiments, the at least one nucleic acid sequence encoding a transcriptional gene silencing element is selected from the group consisting of (i) a silencing nucleic acid which targets the sequence of SEQ ID NO: 1, (ii) a silencing nucleic acid which targets the sequence of SEQ ID NO: 9, (iii) a silencing nucleic acid which targets the sequence of SEQ ID NO: 17; (iv) a silencing nucleic acid which targets a sequence having at least 95% identity with the sequence of SEQ ID NO: 1; (v) a silencing nucleic acid which targets a sequence having at least 95% identity with the sequence of SEQ ID NO: 9; (vi) a silencing nucleic acid which targets a sequence having at least 95% identity with the sequence of SEQ ID NO: 17; (vii) a silencing nucleic acid which targets the sequence of SEQ ID NO: 36; and (viii) a silencing nucleic acid which targets a sequence having at least 95% identity with a sequence of SEQ ID NO: 36.

In some embodiments, the at least one nucleic acid sequence encoding a transcriptional gene silencing element is an RNA duplex comprising a sense strand and an antisense strand, wherein the sense strand comprises a sequence having at least 95% identity to one of the sequence of SEQ ID NO: 6, the sequence of SEQ ID NO: 14, the sequence of SEQ ID NO: 22; or the sequence of SEQ ID NO: 40. In some embodiments, the at least one nucleic acid sequence encoding a transcriptional gene silencing element is an siRNA selected from the group consisting of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; (iii) an siRNA comprising a sense strand having the sequence of SEQ ID NO: 22, and an antisense strand having the sequence of SEQ ID NO: 23; and (iv) an siRNA comprising a sense strand having the sequence of SEQ ID NO: 40, and an antisense strand having the sequence of SEQ ID NO: 41.

In some embodiments, the at least one nucleic acid sequence encoding a transcriptional gene silencing element is an shRNA selected from the group consisting of (i) an shRNA having the sequence of SEQ ID NO: 8; (ii) an shRNA having the sequence of SEQ ID NO: 16; and (iii) an shRNA having the sequence of SEQ ID NO: 24; and (iv) an shRNA having the sequence of SEQ ID NO: 42.

In some embodiments, the nucleic acid sequence that encodes a nucleic acid molecule that inhibits an HIV co-receptor is a siRNA or shRNA having a double-stranded region, wherein a first portion of the double-stranded region comprises a sequence that is identical to part of a sequence of the HIV co-receptor, and wherein a second portion of the double-stranded region comprises a sequence that is complementary to another part of the sequence of the HIV co-receptor. In some embodiments, the HIV co-receptor is CCR5 or CXCR4. In some embodiments, the nucleic acid sequence that encodes a nucleic acid molecule that inhibits an HIV co-receptor is a shRNA which has a sequence of SEQ ID NO: 25. In some embodiments, the inhibitor of the HIV co-receptor is capable of reducing expression of the HIV co-receptor when the vector is expressed in a host cell. In some embodiments, the protein that inhibits HIV fusion to a target cell is a C46 protein. In some embodiments, the protein that inhibits HIV fusion has the sequence of SEQ ID NO: 26. In some embodiments, the protein that inhibits HIV replication is selected from the group consisting of human TRIM5a, rhesus TRIM5a, chimeric TRIM5a, a human TRIM5-cyclophilin fusion protein, cyclophilin, E3 ubiquitin, APOBEC3G, and bone marrow stromal cell antigen 2 (BST-2).

In some embodiments, the expression vector comprises at least two nucleic acids encoding a transcriptional gene silencing element. In some embodiments, the expression vector comprises three nucleic acids encoding a transcriptional gene silencing element. In some embodiments, the expression vector is a viral vector. In some embodiments, the viral vector is a lentiviral vector or a retroviral vector. In some embodiments, the lentiviral vector is self-inactivating. In some embodiments, the expression vector, when expressed in a host cell, confers resistance to infection by (1) X4- and R5-tropic HIV strains, (2) highly active antiretroviral therapy (HAART) resistant HIV strains or (3) X4- and R5-tropic HAART-resistant HIV strains. In some embodiments, each of the nucleic acids are expressed by separate promoters. In some embodiments, least two of the nucleic acids are expressed by a same promoter.

In another aspect of the present disclosure is an expression vector comprising a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV. In some embodiments, the first nucleic acid sequence is operably linked to a first promoter; the second nucleic acid sequence is operably linked to a second promoter; and the third nucleic acid sequence is operably linked to a third promoter. In some embodiments, at least two of the first, second, and third promoters are the same. In some embodiments, the first, second, and third promoters are different. In some embodiments, the expression vector further comprises a fourth nucleic acid, wherein the fourth nucleic acid is selected from the group consisting of another nucleic acid that targets a sequence of the 5′ LTR of HIV, a nucleic acid sequence encoding an inhibitor of HIV replication; and a nucleic acid sequence encoding an inhibitor of viral entry; wherein the four nucleic acid is operably linked to a fourth promoter. In some embodiments, the expression vector comprises at least two silencing nucleic acids that target the 5′LTR of HIV. In some embodiments, the expression vector is a lentiviral vector.

In another aspect of the present disclosure is an expression vector comprising a first nucleic acid encoding an shRNA having a sequence of SEQ ID NO: 25; a second nucleic acid sequence encoding for a C46 protein; and a third nucleic acid sequence encoding a silencing nucleic acid that targets one of a sequence of a 5′ LTR of HIV selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1; wherein the first nucleic acid sequence is operably linked to an H1 pol III promoter, the second nucleic acid sequence is operably linked to a pol II promoters, such as the UbiquitinC pol II promoter, the third nucleic acid sequence operably linked to one of a U6, U6.1, or U6.2 promoter (U6, U6.1, and U6.2 may be collectively referred to as “U6” herein). In some embodiments, the expression vector further comprises a fourth nucleic acid sequence, wherein the fourth nucleic acid sequence is another of a sequence of a 5′ LTR of HIV selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1; and wherein the fourth nucleic acid sequence is operably linked to a U1 promoter. In some embodiments, the expression vector is a lentiviral vector.

In another aspect of the present disclosure is expression vector comprising comprising a first nucleic acid sequence encoding an inhibitory nucleic acid capable of reducing expression of an HIV co-receptor; a second nucleic acid sequence encoding an HIV fusion inhibitor protein, wherein the second nucleic acid is not T20; and a third nucleic acid sequence encoding a silencing nucleic acid that targets a sequence with a 5′ LTR region of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. In some embodiments, the second nucleic acid sequence encoding an HIV fusion inhibitor protein has the sequence of SEQ ID NO: 26.

In another aspect of the present disclosure is a host cell comprising an expression vector comprising a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. In some embodiments, the host cell is a hematopoietic progenitor/stem cell, a monocyte, a macrophage, a peripheral blood mononuclear cell, a CD4+ T lymphocyte, a CD8+ T lymphocyte, or a dendritic cell.

In other aspects of the present disclosure are compositions, such as compositions comprising an expression vector, or compositions comprising an expression vector and another nucleic acid molecule. Thus, in accordance with another aspect of the present disclosure is a composition, including a pharmaceutical composition, comprising an expression vector including a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. In some embodiments, the expression vector is a lentiviral vector. In some embodiments, the composition further comprises a pharmaceutically acceptable carrier. In some embodiments, the composition is formulated as an emulsion. In some embodiments, the composition is formulated with micelles, nanoparticles, or nanocapsules. In some embodiments, the compositions are encapsulated within a polymer. In some embodiments, compositions are encapsulated within liposomes. In some embodiments, the compositions are encapsulated within minicells. In some embodiments, the nucleic acid encoding an HIV fusion inhibitor is other than T20.

In another aspect of the present disclosure is a composition comprising (i) an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) a nucleic acid that encodes at least one transcriptional gene silencing element which targets a sequence of the 5′ LTR of HIV. In some embodiments, the expression vector is a lentiviral vector.

In some embodiments, the nucleic acid that encodes at least one transcriptional gene silencing element is selected from the group consisting of (i) a silencing nucleic acid which targets the sequence of SEQ ID NO: 1, (ii) a silencing nucleic acid which targets the sequence of SEQ ID NO: 9, (iii) a silencing nucleic acid which targets the sequence of SEQ ID NO: 17; (iv) a silencing nucleic acid which targets the sequence of SEQ ID NO: 36; (v) a silencing nucleic acid which targets a sequence having at least 95% identity with the sequence of SEQ ID NO: 1; (vi) a silencing nucleic acid which targets a sequence having at least 95% identity with the sequence of SEQ ID NO: 9; (vii) a silencing nucleic acid which targets a sequence having at least 95% identity with the sequence of SEQ ID NO: 17; and (viii) a silencing nucleic acid which targets a sequence having at least 95% identity with the sequence of SEQ ID NO: 36. In some embodiments, the nucleic acid that encodes at least one transcriptional gene silencing element is an RNA duplex comprising a sense strand and an antisense strand, wherein the sense strand comprises a sequence having at least 95% identity to one of the sequence of SEQ ID NO: 6, the sequence of SEQ ID NO: 14, the sequence of SEQ ID NO: 22, or the sequence or SEQ ID NO: 40. In some embodiments, the nucleic acid that encodes at least one transcriptional gene silencing element is an siRNA selected from the group consisting of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; (iii) an siRNA comprising a sense strand having the sequence of SEQ ID NO: 22, and an antisense strand having the sequence of SEQ ID NO: 23; and (iv) an siRNA comprising a sense strand having the sequence of SEQ ID NO: 40, and an antisense strand having the sequence of SEQ ID NO: 41. In some embodiments, the nucleic acid that encodes at least one transcriptional gene silencing element is an shRNA selected from the group consisting of (i) an shRNA having the sequence of SEQ ID NO: 8; (ii) an shRNA having the sequence of SEQ ID NO: 16; (iii) an shRNA having the sequence of SEQ ID NO: 24; (iv) an shRNA having the sequence of SEQ ID NO: 42. In some embodiments, the composition further comprises a pharmaceutically acceptable carrier.

In yet other aspects of the present disclosure are methods, including (i) methods of inhibiting HIV transcription; (ii) methods of inhibiting HIV replication; (iii) methods of treating an HIV infection; (iv) methods of preventing an HIV infection; (v) methods of reducing an HIV infection; and (vi) methods of preventing and/or reducing a productive HIV infection in a subject not suffering from any HIV infection. These methods are carried out by administering or contacting a cell with an expression vector as described herein, a composition comprising an expression vector and/or other components or active agents, or co-administering a composition comprising (a) an expression vector, and (b) another component or active agent.

Thus, in accordance with another aspect of the present disclosure is a method of inhibiting HIV gene transcription in a cell infected with HIV comprising contacting the cell with an effective amount of the expression vector comprising a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1.

In accordance with another aspect of the present disclosure is a method of inhibiting HIV gene replication in a cell infected with HIV, comprising contacting the cell with an effective amount of the expression vector comprising a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. In some embodiments, the nucleic acid encoding an HIV fusion inhibitor is other than T20.

In another aspect of the present disclosure is a method of treating an HIV infection in a subject, comprising administering to the subject an effective amount of the expression vector comprising a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. Alternatively, the present disclosure also encompasses the use of such an expression vector in the manufacture of a medicament for treating an HIV infection in a subject. In some embodiments, the nucleic acid encoding an HIV fusion inhibitor is other than T20.

In another aspect of the present disclosure is a method of preventing and/or reducing an HIV infection in a subject, comprising administering to the subject an effective amount of the expression vector comprising a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. Alternatively, the present disclosure also encompasses the use of such an expression vector in the manufacture of a medicament for preventing, and/or reducing HIV infection in a subject. In some embodiments, the nucleic acid encoding an HIV fusion inhibitor is other than T20.

In another aspect of the present disclosure is a method of preventing and/or reducing a productive HIV infection in a subject not suffering from an HIV infection, comprising administering to the subject an effective amount of the expression vector comprising a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. Alternatively, the present disclosure also encompasses the use of such an expression vector in the manufacture of a medicament for preventing and/or reducing a productive HIV infection in a subject not suffering from an HIV infection. In some embodiments, the nucleic acid encoding an HIV fusion inhibitor is other than T20.

In another aspect of the present disclosure is a method of inhibiting HIV gene transcription in a cell infected with HIV, comprising contacting the cell with an effective amount of a composition comprising (i) an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) a nucleic acid that encodes at least one transcriptional gene silencing element which targets a sequence of the 5′ LTR of HIV.

In another aspect of the present disclosure is a method of inhibiting HIV gene replication in a cell infected with HIV, comprising contacting the cell with an effective amount of a composition comprising (i) an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) a nucleic acid that encodes at least one transcriptional gene silencing element which targets a sequence of the 5′ LTR of HIV. In some embodiments, the nucleic acid encoding an HIV fusion inhibitor is other than T20.

In another aspect of the present disclosure is a method of treating an HIV infection in a subject, comprising administering to the subject an effective amount of a composition comprising (i) an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) a nucleic acid that encodes at least one transcriptional gene silencing element which targets a sequence of the 5′ LTR of HIV. Alternatively, the present disclosure also encompasses the use of such a composition in the manufacture of a medicament for treating an HIV infection in a subject.

In another aspect of the present disclosure is a method of reducing and/or preventing an HIV infection in a subject, comprising administering to the subject an effective amount of a composition comprising (i) an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) a nucleic acid that encodes at least one transcriptional gene silencing element which targets a sequence of the 5′ LTR of HIV. Alternatively, the present disclosure also encompasses the use of such a composition in the manufacture of a medicament for preventing, and/or reducing HIV infection in a subject.

In another aspect of the present disclosure is a method of preventing and/or reducing a productive HIV infection in a subject not suffering from an HIV infection, comprising administering to the subject an effective amount of a composition comprising (i) an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) a nucleic acid that encodes at least one transcriptional gene silencing element which targets a sequence of the 5′ LTR of HIV. Alternatively, the present disclosure also encompasses the use of such a composition in the manufacture of a medicament for preventing and/or reducing a productive HIV infection in a subject not suffering from an HIV infection.

In another aspect of the present disclosure is a method of treating an HIV infection in a subject comprising co-administering (i) an effective amount of an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) an effective amount of at least one silencing nucleic acid selected from the group consisting of (a) a silencing nucleic acid which targets the sequence of SEQ ID NO: 1, (b) a silencing nucleic acid which targets the sequence of SEQ ID NO: 9, (c) a silencing nucleic acid which targets the sequence of SEQ ID NO: 17; (d) a silencing nucleic acid which targets the sequence of SEQ ID NO: 36; (e) a silencing nucleic acid which targets a sequence having at least 95% identity with the sequence of SEQ ID NO: 1; (f) a silencing nucleic acid which targets a sequence having at least 95% identity with the sequence of SEQ ID NO: 9; and (g) a silencing nucleic acid which targets a sequence having at least 95% identity with the sequence of SEQ ID NO: 17; (h) a silencing nucleic acid which targets a sequence having at least 95% identity with the sequence of SEQ ID NO: 36. In some embodiments, the expression vector is a lentiviral vector. In some embodiments, the co-administering is simultaneous. In some embodiments, the expression vector and the at least one transcriptional gene silencing element are administered at different times. In some embodiments, the expression vector comprises a nucleic acid sequence that encodes a nucleic acid molecule that inhibits an HIV co-receptor; and a nucleic acid sequence that encodes an HIV fusion inhibitor protein. In some embodiments, the HIV co-receptor is CCRS and the HIV fusion inhibitor protein is C46. In some embodiments, the nucleic acid encoding an HIV fusion inhibitor is other than T20.

In another aspect of the present disclosure is a method of reducing and/or preventing an HIV infection in a subject comprising co-administering (i) an effective amount of an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) an effective amount of at least one silencing nucleic acid selected from the group consisting of (a) a silencing nucleic acid which targets the sequence of SEQ ID NO: 1, (b) a silencing nucleic acid which targets the sequence of SEQ ID NO: 9, (c) a silencing nucleic acid which targets the sequence of SEQ ID NO: 17; (d) a silencing nucleic acid which targets the sequence of SEQ ID NO: 36; (e) a silencing nucleic acid which targets a sequence having at least 95% identity with the sequence of SEQ ID NO: 1; (f) a silencing nucleic acid which targets a sequence having at least 95% identity with the sequence of SEQ ID NO: 9; and (g) a silencing nucleic acid which targets a sequence having at least 95% identity with the sequence of SEQ ID NO: 17; (h) a silencing nucleic acid which targets a sequence having at least 95% identity with the sequence of SEQ ID NO: 36. In some embodiments, the expression vector is a lentiviral vector. In some embodiments, the co-administering is simultaneous. In some embodiments, the expression vector and the at least one transcriptional gene silencing element are administered at different times. In some embodiments, the expression vector comprises a nucleic acid sequence that encodes a nucleic acid molecule that inhibits an HIV co-receptor; and a nucleic acid sequence that encodes an HIV fusion inhibitor protein. In some embodiments, the HIV co-receptor is CCRS and the HIV fusion inhibitor protein is C46. In some embodiments, the nucleic acid encoding an HIV fusion inhibitor is other than T20.

In another aspect of the present disclosure is a method of treating an HIV infection in a subject comprising transducing hematopoietic cells with the expression vector, and transplanting the transduced hematopoietic cells in the subject, wherein the transduced hematopoietic cells are resistant to HIV infection, wherein the expression vector comprises a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. In some embodiments, the method comprises transducing hematopoietic cells (e.g., HPSC, CD4+ T lymphocytes, CD8+ T lymphocytes, or monocyte/macrophages) with an expression vector of the disclosure and transplanting the transduced cells in the patient, wherein the transduced cells are resistant to HIV infection. In some embodiments, the hematopoietic cells are hematopoietic progenitor/stem cells (HPSC) that generate granulocytes, monocyte/macrophages, and lymphocytes that are resistant to HIV infection following transplantation into a patient. In some embodiments, the HPSC are autologous and CD34 positive. In some embodiments, the transduced HPSC can generate granulocytes, monocyte/macrophages, and lymphocytes that are resistant to infection by R5 and X4 tropic strains of HIV. In some embodiments, the transduced HPSC can generate granulocytes, monocyte/macrophages, and lymphocytes that are resistant to infection by HIV strains that are resistant to cART. Alternatively, the present disclosure also encompasses the use of such cells in the manufacture of a medicament for treating and/or preventing an HIV infection in a subject.

In another aspect of the present disclosure is a method of preventing and/or reducing an HIV infection in a subject comprising transducing hematopoietic cells with the expression vector, and transplanting the transduced hematopoietic cells in the subject, wherein the transduced hematopoietic cells are resistant to HIV infection, wherein the expression vector comprises a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. In some embodiments, the method comprises transducing hematopoietic cells (e.g., HPSC, CD4+ T lymphocytes, CD8+ T lymphocytes, or monocyte/macrophages) with an expression vector of the disclosure and transplanting the transduced cells in the patient, wherein the transduced cells are resistant to HIV infection. In some embodiments, the hematopoietic cells are hematopoietic progenitor/stem cells (HPSC) that generate granulocytes, monocyte/macrophages, and lymphocytes that are resistant to HIV infection following transplantation into a patient. In some embodiments, the HPSC are autologous and CD34 positive. In some embodiments, the transduced HPSC can generate granulocytes, monocyte/macrophages, and lymphocytes that are resistant to infection by R5 and X4 tropic strains of HIV. In some embodiments, the transduced HPSC can generate granulocytes, monocyte/macrophages, and lymphocytes that are resistant to infection by HIV strains that are resistant to cART. Alternatively, the present disclosure also encompasses the use of such cells in the manufacture of a medicament for treating and/or preventing an HIV infection in a subject. In some embodiments, the nucleic acid encoding an HIV fusion inhibitor is other than T20.

In another aspect of the present disclosure is a method of making the expression vectors described herein as well as pharmaceutical compositions comprising the novel expression vectors. In some embodiments, the method of producing a viral expression vector which, when present in a cell, is capable of inhibiting binding of HIV to the cell and preventing HIV fusion into the cell or HIV replication, comprises synthesizing a cDNA of a gene which expresses a protein capable of preventing HIV fusion into a cell or HIV replication; cloning the synthesized cDNA into a restriction site in a viral vector; and inserting an expression unit capable of down regulating expression of an HIV co-receptor into a restriction site in the vector.

Yet another aspect of the disclosure provides for a kit comprising an expression vector of the present disclosure. The kit may be combined with another active pharmaceutical ingredient for administration to a subject in need thereof.

The present disclosure is based, in part, on the recognition that a therapeutic approach that targets non-HIV genes and/or proteins (i.e. host cell genes and/or proteins) decreases the probability that new HIV strains resistant to the inhibitors will emerge. In untreated CD34+ hematopoietic stem cells (HPSC) and/or CD4+ T cells, HIV binds to CD4 and CCR5 and then fuses with the cell membrane. Following fusion, the viral RNA is introduced and, subsequent to reverse transcription to cDNA that becomes double stranded, it integrates into the host cell genome where it is transcribed to produce more HIV. In the (combination) treated CD34+ HSC and/or CD4+ T cells, HIV is blocked from binding to CCR5 and from fusion with the cell membrane. Without wishing to be bound by any particular theory, it is believed that if HIV overcomes these two blocks and viral RNA is introduced and integrates into the host cell genome as DNA, transcription is blocked as the transcriptional gene silencing (TGS)-inducing shRNA, included within the expression vectors of the present disclosure, causes the HIV DNA to be transcriptionally inactive.

Moreover, and in view of the teachings of Trobridge, the ordinary-skilled artisan would have been led away from including a nucleic acid sequence encoding a fusion inhibitor protein in the same vector as a nucleic acid sequence encoding an inhibitory nucleic acid molecule, let alone including a third nucleic acid sequence encoding a transcriptional gene silencing element. The skilled person would have been led away simply because Trobridge explicitly indicates that dual combination vectors were actually less effective in inhibiting HIV replication than vectors encoding the fusion inhibitor protein alone. There, the very low levels of gene transfer to hematopoietic cells exhibited by the dual vector would have also discouraged one of ordinary skill in the art from modifying a vector encoding a single nucleic acid to incorporate additional nucleic acid sequences because such low gene transfer levels have been the primary reason for failure of gene therapies in clinical trials. Given this, the skilled artisan reading Trobridge would certainly have expected a triple combination vector or a quadruple combination vector, such as disclosed herein, to not work and certainly be less efficacious than any single vector or dual vector systems.

Applicants submit that the present disclosure provides for superior results as compared with Trobridge. In fact, Applicants have demonstrated the ability to stably introduce LVsh5/C46 (also referred to herein as “Cal-1”) into various hematopoietic cells, including CD4+ T lymphocytes and CD34+ HSPC, to allow for expression of a CCR5-targeted shRNA (sh5) and C46. In fact, Applicants have demonstrated that treatment of target cells with the LVsh5/C46 vector did not show any indication of toxicity. (See, for example, Wolstein et al., “Preclinical Safety and Efficacy Of An Anti-HIV-1 Lentiviral Vector Containing A Short Hairpin RNA To CCR5 and The C46 Fusion Inhibitor,” Molecular Therapy—Methods & Clinical Development (2014) 1, 11; doi:10.1038/mtm.2013.11; see also Burke, et al. “Engineering Cellular Resistance To HIV-1 Infection In Vivo Using A Dual Therapeutic Lentiviral Vector,” Molecular Therapy—Nucleic Acids (2015) 4, e236; doi:10.1038/mtna.2015.10, the disclosures of which are hereby incorporated by reference herein).

And, as further noted herein, highly active antiretroviral therapy (e.g. cART) uses two to three active pharmaceutical ingredients to effectively suppress HIV replication and mitigate the development of resistance. Applicants believe that a triple or quadruple combination vector, such as disclosed herein, may likewise provide an efficacious means for treatment and/or suppression of HIV. Accordingly, and without wishing to be bound by any particular theory, Applicants believe that a triple or quadruple combination vector may be as safe as dual vector constructs, while providing comparatively superior results.

### BRIEF DESCRIPTION OF THE DRAWINGS

FIG. 1 illustrates how the novel combination vector of the present disclosure may inhibit HIV. Model of mechanisms targeted by complex LV construct in protection against HIV infection.

FIG. 2 illustrates the position of the TGS-inducing si/shRNAs. Model of the 5′LTR including positions of Nuc-0 and Nuc-1, and target sequences of our lead candidate si/shRNAs. These target the sequence within the tandem NF-kB binding sites (PromA) and a sequence upstream (si143), in the region of nuc-0. These induce similar epigenetic changes. We have shown these molecules act in the nucleus through Ago1 recruitment followed by HDAC 1 and 2s and HMTs e.g. Enhancer of Zeste.

FIG. 3 shows siRNA 143, 136, 205 and PromA target sequence conservation in the HIV-1 5′LTR promoter across subtypes A-U.

FIG. 4 shows self-inactivating lentiviral vectors containing modified LTRs allowing integration but not expression of viral genome, based on the Calimmune Cal-1 backbone vector (see US Publication No. US2012/0201794, the disclosure of which is incorporated by reference herein), consisting of H1, H1 promoter and/or U6, U6 promoter and/or U1, U1 promoter; sh5, short hairpin RNA CCR5; or shPrA, short hairpin RNA PromA; and/or sh143, short hairpin RNA 143; UbC, ubiquitin C promoter; and C46, C46 fusion inhibitor. To measure construct expression all modified constructs contain EGFP, enhanced green fluorescent protein.

FIG. 5A is a diagram showing the region within HIV-1 5′LTR targeted by the 143, 136, 205 and PromA siRNAs. Arrow indicates transcription start site, underlined sequence indicates tandem repeat NF-κB binding sites, siPromA-M2 and si143T are inactive mutants, used as specificity controls.

FIG. 5B is a graph Si136T-, si143-, si205S- and siPromA-transfected cultures show potent suppression of HIV-1SF162 virus transcription 15 days post-infection compared to inactive mutant controls. *siRNA target identical to HIV-1NL4.3 strain. **siRNA target identical to HIV-1SF162 strain.

FIGS. 6A-6G show heterochromatin marks observed in siRNA suppressed HIV-1 cultures. To confirm the suppressive effect of the LTR targeting siRNAs was occurring through transcriptional gene silencing (TGS) mechanisms, chromatin immunoprecipitation (ChIP) assays were performed in HeLa T4+ cultures with suppressed HIV-1 following siRNA 143, 136, 205 and PromA transfection or cultures with active HIV transcription following mock-transfection. Heterochromatin marks consisted with TGS were observed at day 6 post-infection in siRNA 143, 136, 205 and PromA-transfected cultures, which included enrichment of histone 3 lysine 27 trimethylation (H3K27me3), and reduction in histone 3 lysine 9 acetylation (H3K9Ac), as compared to mock-transfected cultures. ChIP analysis of heterochromatin marks also showed enrichment of H3K9me3 and recruitment of Ago1 in si143- and siPromA-transfeced cells.

FIG. 7 provides a graph showing robust resistance to reactivation stimuli by shRNA. To investigate the effect of the siRNA 143 target sequence in an HIV-1 latency model, J-Lat 9.2 cells were stably transduced with short hairpin (sh)143 and/or shPromA using lentivirus vectors. Reactivation of integrated latent HIV-1 was attempted in J-Lat 9.2 cells using SAHA, TNF or a combination of SAHA/TNF at various concentrations and GFP expression was measured as a read out of HIV transcription, at 48 h. J-Lat 9.2 cells transduced with sh143 and/or shPromA were observed to be largely resistant to reactivation from SAHA, TNF or combinations of SAHA/TNF at physiological concentrations (Cillo et al., 2014 PNAS; De Pablo-Bernal et al., 2014 J Antimicrob Chemother) and showed low level reactivation even at supra-physiological drug concentrations, while those transduced with a shRNA control showed highly elevated GFP expression, indicating reactivation of latent HIV-1 infection. Importantly, the dual sh143/shPromA transduced J-Lat 9.2 cell line appeared to show slightly better protection against reactivation stimuli. These data demonstrate that the HIV-1 latency model J-Lat 9.2 cells are largely resistant to reactivation by drug treatments when transduced with sh143 and/or shPromA. These findings are important in the context of gene therapy applications, where patients could be exposed to various immune and inflammatory stimuli.

FIG. 8A provides a graph setting forth an analysis of intracellular HIV-1 DNA.

FIG. 8B provides a graph setting forth an analysis of HIV-1 specific LTR mRNA.

FIG. 9A provides a graph setting forth an analysis of HIV-1 integrated DNA in PM-1 cells.

FIG. 9B provides a graph setting forth an analysis of HIV-1 specific gag mRNA.

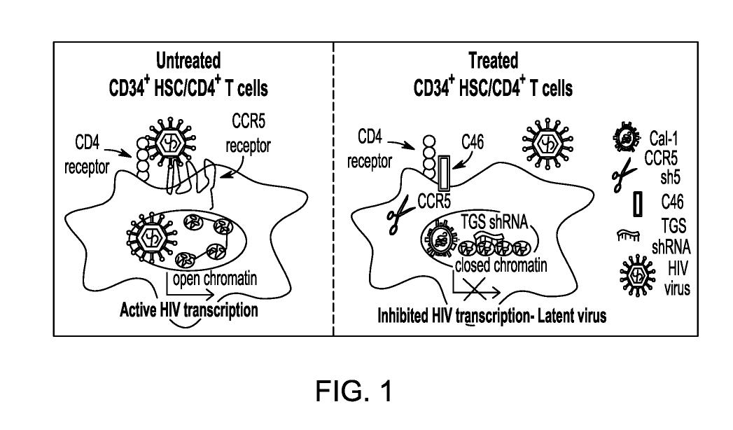

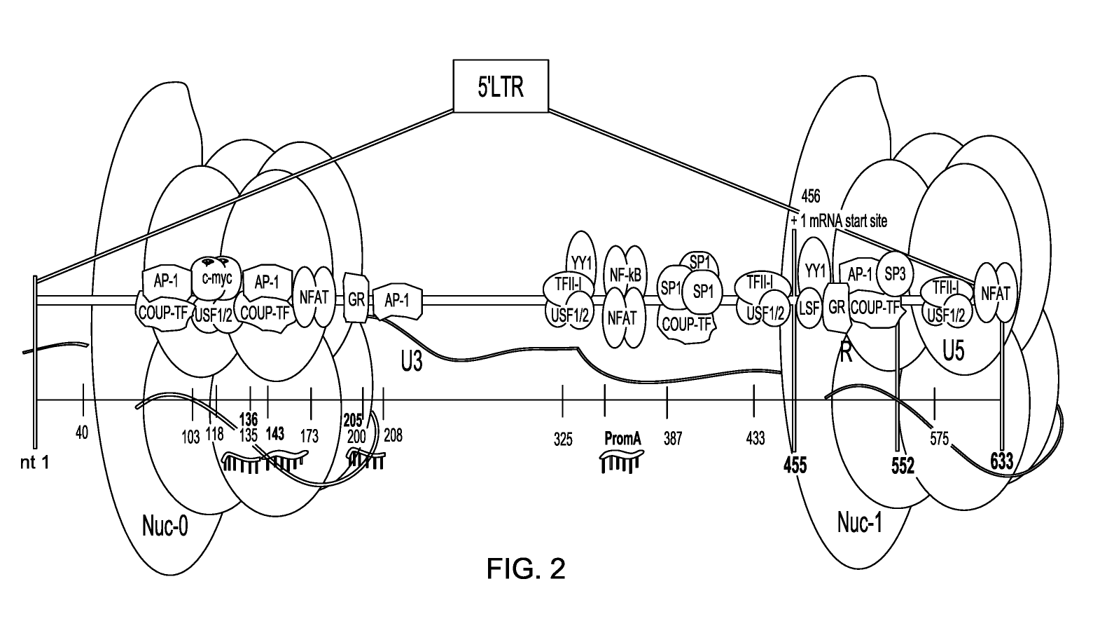

### SEQUENCE LISTING

The nucleic and amino acid sequences provided herein are shown using standard letter abbreviations for nucleotide bases, and three letter code for amino acids, as defined in 37 C.F.R. 1.822. The sequence listing is submitted as an ASCII text file, named “2016-02-11_Cal-002WO_ST25.txt” created on Feb. 11, 2016, 5 KB, which is incorporated by reference herein.

### DETAILED DESCRIPTION

In general, the present disclosure provides expression vectors, compositions comprising expression vectors, and methods of treating subjects by administering such expression vectors or compositions.

As used herein, the singular terms “a,” “an,” and “the” include plural referents unless the context clearly indicates otherwise. Similarly, the word “or” is intended to include “and” unless the context clearly indicates otherwise.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of ” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, a “cell infected with HIV” refers to a cell in which the HIV genome has integrated into the cell genome, and includes cells producing HIV virus, and cells latently infected with HIV. As used herein, a cell latently infected with HIV is a cell in which the HIV genome is integrated into the host cell genome, but which is transcriptionally inactive but capable of reactivation to a transcriptionally active state.

As used herein, “contacting the cell” refers to bringing a composition, formulation, or agent into contact with a cell in a manner which produces an effective result (i.e. reducing, mitigating, or eliminating HIV infection, transcription, or replication). Contacting the cell also includes introducing a composition, formulation, or agent into a cell.

As used herein, the phrases “inhibiting HIV transcription” refers to reducing or preventing transcription of one or more HIV genes to the extent that infectious HIV virus particle formation in the cell is reduced, mitigated, or eliminated.

As used herein, the phrases “inhibiting HIV replication” refers to reducing or preventing replication of one or more HIV genes to the extent that infectious HIV virus particle formation in the cell is reduced, mitigated, or eliminated.

As used herein, the term “HIV” includes not only HIV-1, but also the various strains of HIV-1 (e.g. strain BaL or strain SF162) and the various subtypes of HIV-1 (e.g. subtypes A, B, C, D, F, G H, J, and K).

As used herein, “polynucleotide” refers to single or double stranded DNA, RNA, or modified versions thereof including peptide nucleic acid and locked nucleic acid (LNA).

As used herein, a “silencing nucleic acid” refers to any polynucleotide which is capable of interacting with a specific sequence to inhibit gene expression. Examples of silencing nucleic acids include RNA duplexes (e.g. siRNA, shRNA), LNAs, antisense RNA, DNA polynucleotides which encode sense and/or antisense sequences of the siRNA or shRNA, DNAzymse, or ribozymes. The inhibition of gene expression need not necessarily be gene expression from a specific enumerated sequence, and may be, for example, gene expression from a sequence controlled by that specific sequence.

As used herein “transcriptional gene silencing elements” are silencing nucleic acid sequences which target a sequence within the region of the 5′ long terminal repeat (LTR) of HIV (including HIV-1).

Expression Vectors

The present disclosure provides expression vectors comprising at least three nucleic acid sequences, where the at least three nucleic acids are selected from the group consisting of nucleic acid sequences encoding transcriptional gene silencing elements which target a sequence of the 5′ LTR of HIV; nucleic acid sequences that encode inhibitors of an HIV co-receptor; nucleic acid sequences that encode proteins that inhibit HIV fusion to a target cell; nucleic acid sequences that encode proteins that inhibit HIV replication; and nucleic acid sequences that encode an inhibitor of viral entry. As used herein, “expression vector” or “vector” refers to a composition of matter which can be used to deliver nucleic acids of interest to the interior of a cell such that they will be expressed by the cell. Numerous vectors are known in the art including, but not limited to, linear polynucleotides, polynucleotides associated with ionic or amphiphilic compounds, plasmids, and viral vectors. Examples of viral vectors include, but are not limited to, adenoviral vectors, adeno-associated virus vectors, retroviral vectors (including lentiviral vectors), and the like. In one embodiment, the expression vector is a viral vector. Preferably, the viral vector is a retroviral or lentiviral vector.

In other embodiments is expression vector comprising at least first, second, and third nucleic acid sequences, wherein the first nucleic acid sequence comprises a nucleic acid sequence encoding a transcriptional gene silencing element; wherein the second and third nucleic acid sequences are selected from the group consisting of a nucleic acid sequence that encodes an inhibitor of an HIV co-receptor, a nucleic acid sequence that encodes a protein that inhibits HIV fusion to a target cell, a nucleic acid sequence that encodes a protein that inhibits HIV replication, and a nucleic acid sequence that encodes an inhibitor of viral entry; and wherein the second and third nucleic acid sequences are different.

In yet other embodiments are expression vectors comprising (i) at least one nucleic acid sequence encoding a transcriptional gene silencing element; and (ii) at least two other nucleic acid sequences selected from the group consisting of a nucleic acid sequence that encodes an inhibitor of an HIV co-receptor; a nucleic acid sequence that encodes a protein that inhibits HIV fusion to a target cell; a nucleic acid sequence that encodes a protein that inhibits HIV replication; and a nucleic acid sequence that encodes an inhibitor of viral entry. In some embodiments, each of the nucleic acid sequences are expressed from different promoters on the expression vector. In some embodiments, each of the promoters can be the same or different from one another. For example, the at least one nucleic acid sequence encoding a transcriptional gene silencing element may be expressed from a first promoter; and each of the at least two other nucleic acid sequences are themselves expressed from separate promoters (e.g. second and third promoters). In other embodiments, two of the three nucleic acid sequences are transcribed from a single promoter (i.e. the first and second nucleic acid sequences or the second and third nucleic acid sequences). In yet other embodiments, three nucleic acid sequences are transcribed from a single promoter.

In further embodiments, each of the promoters (e.g. first, second, third, and optionally fourth promoters) can be RNA polymerase I (pol I), polymerase II (pol II), or polymerase III (pol III) promoters. The promoters may be constitutive promoters or inducible promoters as known to those of ordinary skill in the art. In some embodiments, the promoter contains at least a portion of an HIV LTR (e.g. TAR) and is inducible by HIV infection. In certain embodiments, the first promoter is a RNA pol III promoter. RNA pol III promoters suitable for use in the expression vectors of the disclosure include, but are not limited, to human U6, mouse U6, and human H1 others. In one embodiment, the first promoter is a H1 RNA pol III promoter. In other embodiments, the second promoter is a RNA pol II promoter. In one particular embodiment, the second promoter is a UbiquitinC pol II promoter. The second promoter, in some embodiments, can be a tissue-specific promoter. For instance, suitable tissue-specific promoters include macrophage-specific promoters (e.g., MPG-1 and the like) and T-cell promoters (e.g., CD4 and the like). In one embodiment, the third promoter is a RNA pol II promoter. In another embodiment, the third promoter is a UbiquitinC pol II promoter. The third promoter can, in some embodiments, be a tissue specific promoter. The first, second, and third promoters can be a combination of any of the promoters described herein. In certain embodiments, RNA pol III promoters are preferred where the nucleic acid sequence encodes an inhibitory RNA molecule, such as an siRNA or shRNA. In other embodiments, RNA pol II promoters are preferred where the nucleic acid sequence encodes a protein.

In some embodiments, the expression vector is a viral vector. In some embodiments, the viral vector is a lentiviral vector, as described herein. In other embodiments, the viral vector is a retroviral vector, also described herein.

“Retroviruses” are viruses having an RNA genome that is reverse transcribed by retroviral reverse transcriptase to a cDNA copy that is integrated into the host cell genome. Retroviral vectors and methods of making retroviral vectors are known in the art. Briefly, to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., Cell, Vol. 33:153-159, 1983). When a recombinant plasmid containing a cDNA, together with the retroviral LTR and packaging sequences, is introduced into this cell line, the packaging sequence allows the RNA transcript of the recombinant plasmid to be packaged into viral particles, which are then secreted into the culture media. The media containing the recombinant retroviruses is then collected, optionally concentrated, and used for gene transfer.

“Lentivirus” refers to a genus of retroviruses that is capable of infecting dividing and non-dividing cells. Several examples of lentiviruses include HIV (human immunodeficiency virus: including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which causes immune deficiency and encephalopathy in sub-human primates.

A lentiviral genome is generally organized into a 5′ long terminal repeat (LTR), the gag gene, the pol gene, the env gene, the accessory genes (nef, vif, vpr, vpu) and a 3′ LTR. The viral LTR is divided into three regions called U3, R and U5. The U3 region contains the enhancer and promoter elements. The U5 region contains the polyadenylation signals. The R (repeat) region separates the U3 and U5 regions and transcribed sequences of the R region appear at both the 5′ and 3′ ends of the viral RNA. See, for example, “RNA Viruses: A Practical Approach” (Alan J. Cann, Ed., Oxford University Press, (2000)); O Narayan and Clements (1989) J. Gen. Virology, Vol. 70:1617-1639; Fields et al. (1990) Fundamental Virology Raven Press.; Miyoshi H, Blamer U, Takahashi M, Gage F H, Verma I M. (1998) J Virol., Vol. 72(10):8150 7, and U.S. Pat. No. 6,013,516.

Lentiviral vectors are known in the art, including several that have been used to infect hematopoietic progenitor/stem cells (HPSC). Such vectors can be found, for example, in the following publications, which are incorporated herein by reference: Evans et al., Hum Gene Ther., Vol. 10:1479-1489, 1999; Case et al., Proc Natl Acad Sci USA, Vol. 96:2988-2993, 1999; Uchida et al., Proc Natl Acad Sci USA, Vol. 95:11939-11944, 1998; Miyoshi et al., Science, Vol. 283:682-686, 1999; and Sutton et al., J. Virol., Vol. 72:5781-5788, 1998. In one embodiment, the expression vector is a modified lentivirus, and thus is able to infect both dividing and non-dividing cells. Such lentiviral vectors comprise a modified lentiviral genome that comprises a first nucleic acid sequence encoding an inhibitor of an HIV co-receptor and a second nucleic acid sequence encoding a protein that inhibits HIV fusion to a target cell or HIV replication. Further, the modified lentiviral genome preferably lacks genes for lentiviral proteins required for viral replication, thus preventing undesired replication, such as replication in the target cells. The required proteins for replication of the modified genome are preferably provided in trans in the packaging cell line during production of the recombinant retrovirus (or specifically lentivirus). In one embodiment, the packaging cell line is a 293T cell line. The lentiviral vector preferably comprises sequences from the 5′ and 3′ long terminal repeats (LTRs) of a lentivirus. In one embodiment, the viral construct comprises the R and U5 sequences from the 5′ LTR of a lentivirus and an inactivated or self-inactivating 3′ LTR from a lentivirus. The LTR sequences may be LTR sequences from any lentivirus including from any species or strain. For example, the LTR may be LTR sequences from HIV, simian immunodeficiency virus (SIV), feline immunodeficiency virus (FIV) or bovine immunodeficiency virus (BIV). Preferably the LTR sequences are HIV LTR sequences.

Transcriptional Gene Silencing Elements

In some embodiments, the expression vectors of the present disclosure comprise one or more transcriptional gene silencing elements which target a sequence of the 5′ LTR of HIV. In some embodiments, the expression vectors comprise at least two of the silencing nucleic acids which target a sequence of the 5′ LTR of HIV. Without wishing to be bound by any particular theory, it is believed that the silencing nucleic acids that target a region within the 5′ LTR sequence of HIV described herein are effective at inhibiting transcription of the HIV genome, and therefore represent a method by which HIV replication can be inhibited. Throughout this disclosure, the numbering of the silencing nucleic acids is based on the 5′LTR U3 start site in the subtype B HXB2 strain (Accession no. K03455) (Wong-Staal, F. 1985, Nature 313:277-284).

In some embodiments, the silencing nucleic acid targets a sequence having at least 95% identity with a target sequence from about position 143 to about position 161 of the 5′ LTR of HIV-1. In some embodiments, the silencing nucleic acid targets a sequence having at least 95% identity with that of SEQ ID NO: 1. In other embodiments, the silencing nucleic acid targets a sequence having at least 97% identity with that of SEQ ID NO: 1. In further embodiments, the silencing nucleic acid targets a sequence having at least 99% identity with that of SEQ ID NO: 1. In yet further embodiments, the silencing nucleic acid targets a sequence having about 100% identity with that of SEQ ID NO: 1.

In some embodiments, the silencing nucleic acid targets a sequence having at least 95% identity with a target sequence from about position 136 to about position 154 of the 5′ LTR of HIV-1. In some embodiments, the silencing nucleic acid targets a sequence having at least 95% identity with that of SEQ ID NO: 9. In yet other embodiments, the silencing nucleic acid targets a sequence having at least 97% identity with that of SEQ ID NO: 9. In further embodiments, the silencing nucleic acid targets a sequence having at least 99% identity with that of SEQ ID NO: 9. In yet further embodiments, the silencing nucleic acid targets a sequence having about 100% identity with that of SEQ ID NO: 9.

In some embodiments, the silencing nucleic acid targets a sequence having at least 95% identity with a target sequence from about position 205 to about position 223 of the 5′ LTR of HIV-1. In some embodiments, the silencing nucleic acid targets a sequence having at least 95% identity with that of SEQ ID NO: 17. In other embodiments, the silencing nucleic acid targets a sequence having at least 97% identity with that of SEQ ID NO: 17. In further embodiments, the silencing nucleic acid targets a sequence having at least 99% identity with that of SEQ ID NO: 17. In yet further embodiments, the silencing nucleic acid targets a sequence having about 100% identity with that of SEQ ID NO: 17.

In some embodiments, the silencing nucleic acid targets a sequence having at least 95% identity with a target sequence from about position 350 to about position 368 of the 5′ LTR of HIV-1. In some embodiments, the silencing nucleic acid targets a sequence having at least 95% identity with that of SEQ ID NO: 36. In other embodiments, the silencing nucleic acid targets a sequence having at least 97% identity with that of SEQ ID NO: 36. In further embodiments, the silencing nucleic acid targets a sequence having at least 99% identity with that of SEQ ID NO: 36. In yet further embodiments, the silencing nucleic acid targets a sequence having about 100% identity with that of SEQ ID NO: 36.

As will be appreciated by those skilled in the art, there may exist variation in the sequence of the 5′ LTR region between strains or subtypes of HIV-1, and thus there may be some variation in the target sequence of HIV-1. In some embodiments, the silencing nucleic acid targets one or more sequences selected from SEQ ID NO: 2, SEQ ID NO: 3, SEQ ID NO: 4, or SEQ ID NO: 5, or a sequence having at least 95% identity to one of SEQ ID NO: 2, SEQ ID NO: 3, SEQ ID NO: 4, or SEQ ID NO: 5. In other embodiments, the silencing nucleic acid targets one or more sequences selected from SEQ ID NO: 10, SEQ ID NO: 11, SEQ ID NO: 12, or SEQ ID NO: 13, or a sequence having at least 95% identity to one of SEQ ID NO: 10, SEQ ID NO: 11, SEQ ID NO: 12, or SEQ ID NO: 13. In yet other embodiments, the silencing nucleic acid targets one or more sequences selected from SEQ ID NO: 18, SEQ ID NO: 19, SEQ ID NO: 20, SEQ ID NO: 21, or SEQ ID NO 33, or a sequence having at least 95% identity to one of SEQ ID NO: 18, SEQ ID NO: 19, SEQ ID NO: 20, SEQ ID NO: 21, or SEQ ID NO 33. In further embodiments, the silencing nucleic acid targets one or more sequences selected from SEQ ID NO: 37, SEQ ID NO: 38, or SEQ ID NO 39, or a sequence having at least 95% identity to one of SEQ ID NO: 37, SEQ ID NO: 38, or SEQ ID NO 39.

In some embodiments, the silencing nucleic acid which targets the sequence from about position 143 to about position 161 of the 5′ LTR of HIV-1 is an RNA duplex comprising a sense strand and an antisense strand, wherein the sense strand comprises a sequence having at least 95% identity to that of SEQ ID NO: 6. In other embodiments, the silencing nucleic acid is an RNA duplex comprising a sense strand and an antisense strand, wherein the sense strand comprises a sequence having at least 97% identity to that of SEQ ID NO: 6. In further embodiments, the silencing nucleic acid is an RNA duplex comprising a sense strand and an antisense strand, wherein the sense strand comprises a sequence having at least 99% identity to that of SEQ ID NO: 6. In yet further embodiments, the silencing nucleic acid is an RNA duplex comprising a sense strand and an antisense strand, wherein the sense strand comprises a sequence having about 100% identity to that of SEQ ID NO: 6.

In some embodiments, the silencing nucleic acid which targets the sequence from about position 136 to about position 154 of the 5′ LTR of HIV-1 is an RNA duplex comprising a sense strand and an antisense strand, wherein the sense strand comprises a sequence having at least 95% identity to that of SEQ ID NO: 14. In other embodiments, the silencing nucleic acid is an RNA duplex comprising a sense strand and an antisense strand, wherein the sense strand comprises sequence having at least 97% identity to that of SEQ ID NO: 14. In further embodiments, the silencing nucleic acid is an RNA duplex comprising a sense strand and an antisense strand, wherein the sense strand comprises a sequence having at least 99% identity to that of SEQ ID NO: 14. In yet further embodiments, the silencing nucleic acid is an RNA duplex comprising a sense strand and an antisense strand, wherein the sense strand comprises a sequence having about 100% identity to that of SEQ ID NO: 14.

In some embodiments, the silencing nucleic acid which targets the sequence from about position 205 to about position 223 of the 5′ LTR of HIV-1 is an RNA duplex comprising a sense strand and an antisense strand, wherein the sense strand comprises a sequence having at least 95% identity to that of SEQ ID NO: 22. In other embodiments, the silencing nucleic acid is an RNA duplex comprising a sense strand and an antisense strand, wherein the sense strand comprises sequence having at least 97% identity to that of SEQ ID NO: 22. In further embodiments, the silencing nucleic acid is an RNA duplex comprising a sense strand and an antisense strand, wherein the sense strand comprises a sequence having at least 99% identity to that of SEQ ID NO: 22. In yet further embodiments, the silencing nucleic acid is an RNA duplex comprising a sense strand and an antisense strand, wherein the sense strand comprises a sequence having about 100% identity to that of SEQ ID NO: 22.

In some embodiments, the silencing nucleic acid which targets the sequence having at least 95% identity with a target sequence from about position 350 to about position 368 of the 5′ LTR of HIV-1 is an RNA duplex comprising a sense strand and an antisense strand, wherein the sense strand comprises a sequence having at least 95% identity to that of SEQ ID NO: 40. In other embodiments, the silencing nucleic acid is an RNA duplex comprising a sense strand and an antisense strand, wherein the sense strand comprises sequence having at least 97% identity to that of SEQ ID NO: 40. In further embodiments, the silencing nucleic acid is an RNA duplex comprising a sense strand and an antisense strand, wherein the sense strand comprises a sequence having at least 99% identity to that of SEQ ID NO: 40. In yet further embodiments, the silencing nucleic acid is an RNA duplex comprising a sense strand and an antisense strand, wherein the sense strand comprises a sequence having about 100% identity to that of SEQ ID NO: 40.

The antisense strand of any RNA duplex comprises a sequence that is complementary to the sense strand. As used herein, a “sequence that is complementary to the sense strand” will be able to form a duplex despite having a less than 100% complementarity to the sense strand if at least a portion of the sequence is able to form a duplex with the sense strand. In some embodiments, the antisense strand is at least 95% complementary to the sense strand. In other embodiments, the antisense strand is at least 97% complementary to the sense strand. In further embodiments, the antisense strand is at least 99% complementary to the sense strand. In yet further embodiments, the antisense strand is about 100% complementary to the sense strand. It will be appreciated that the degree of identity between the sense and antisense strands of the RNA duplex may be different than the degree of identity between the sense strand and the respective target sequence.

The two strands forming the RNA duplex may be different portions of one larger RNA molecule, or they may be separate RNA molecules. Where the strands of the RNA duplex are formed from separate RNA molecules, the RNA duplex may be a “small interfering RNA” (“siRNA”). A “small interfering RNA” or “siRNA” is a double-stranded RNA molecule that is capable of inhibiting the expression of a gene with which it shares homology. The region of the gene or other nucleotide sequence over which there is homology is known as the “target region.” Where the two strands are part of one larger molecule, and therefore are connected by a chain of nucleotides between the 3′-end of one strand and the 5′ end of the other strand of the RNA duplex, the RNA duplex is a “short hairpin RNA” (“shRNA”).

In some embodiments, the RNA duplex is a siRNA comprising a sense strand and an antisense strand. In one embodiment, the RNA duplex is a siRNA having a sense strand having the sequence of SEQ ID NO: 6, and an antisense strand having the sequence of SEQ ID NO: 7 (referred to herein as “si143” or “siRNA143”). In another embodiment, the RNA duplex is a siRNA having a sense strand having the sequence of SEQ ID NO: 14, and an antisense strand having the sequence of SEQ ID NO: 15 (referred to herein as “si136” or “siRNA136”). In a further embodiment, the RNA duplex is a siRNA having a sense strand having the sequence of SEQ ID NO: 22, and an antisense strand having the sequence of SEQ ID NO: 23 (referred to herein as “si205” or “siRNA205”). In yet a further embodiment, the RNA duplex is a siRNA having a sense strand having the sequence of SEQ ID NO: 40, and an antisense strand having the sequence of SEQ ID NO: 41 (referred to herein as “siRNA PromA” or “siPromA”).

In other embodiments, the RNA duplex is a shRNA comprising a sense strand and an antisense strand. In one embodiment, the RNA duplex is a shRNA comprising a sense strand having the sequence of SEQ ID NO: 6 (or a sense strand having at least 95% identity to that of SED IQ NO: 6), and an antisense strand having the sequence of SEQ ID NO: 7. In yet another embodiment, the shRNA has the sequence of SEQ ID NO: 8 (referred to herein as “sh143” or “shRNA143”). In another embodiment, the RNA duplex is a shRNA comprising a sense strand having the sequence of SEQ ID NO: 14 (or a sense strand having at least 95% identity to that of SED IQ NO: 14), and an antisense strand having the sequence of SEQ ID NO: 15. In yet another embodiment, the shRNA has the sequence SEQ ID NO: 16 (referred to herein as “sh136” or “shRNA136”). In a further embodiment, the RNA duplex is a shRNA comprising a sense strand having the sequence of SEQ ID NO: 22 (or a sense strand having at least 95% identity to that of SED IQ NO: 22), and an antisense strand having the sequence of SEQ ID NO: 23. In yet another embodiment, the shRNA has the sequence of SEQ ID NO: 24 (referred to herein as “sh205” or “shRNA205”). In yet a further embodiment, the RNA duplex is a shRNA comprising a sense strand having the sequence of SEQ ID NO: 40 (or a sense strand having at least 95% identity to that of SED IQ NO: 40), and an antisense strand having the sequence of SEQ ID NO: 41. In yet another embodiment, the shRNA has the sequence of SEQ ID NO: 42 (referred to herein as “shPromA” or “shRNA PromA”).

In other embodiments, the at least one silencing nucleic acid is a nucleic acid selected from the group consisting of antisense RNA, DNA or mixtures thereof; a DNAzyme; and a ribozyme; which target at least one of a sequence of SEQ ID NO: 1, a sequence of SEQ ID NO: 9, a sequence of SEQ ID NO: 17, or a sequence of SEQ ID NO: 36. Methods for the preparation and use of antisense RNA and DNA molecules, ribozymes and DNAzymes are known in the art and are described in, for example, Jakobsen et al, 2007, Retrovirology 4: 29-41, the disclosure of which is hereby incorporated herein by reference in its entirety.

In other embodiments, the silencing nucleic acid may be a DNA polynucleotide which encodes the sense and/or antisense sequence of the siRNA or shRNA. Such DNA sequences may be inserted into vectors, such as plasmids, viral vectors, etc., to achieve expression of the siRNA or shRNA in the cell. In the case of siRNA, the sense and antisense strands of the siRNA may be expressed from the same or different vectors.

Nucleic Acid Sequences which Encode Inhibitors of an HIV Co-Receptor

In some embodiments, the inhibitor of an HIV co-receptor is an inhibitory nucleic acid, including a siRNA, a shRNA, an aptamer, a ribozyme, and an antisense oligonucleotide. Thus, in some embodiments, a nucleic acid sequence of an expression vector encodes an inhibitory nucleic acid that targets an HIV co-receptor. “Target” refers to the ability of the inhibitor to bind to and/or interfere with an endogenous transcript encoding the HIV co-receptor. For instance, the inhibitory nucleic acid can have a sequence that is substantially complementary to a nucleic acid encoding the HIV co-receptor such that the inhibitory nucleic acid binds to the HIV co-receptor-encoding nucleic acid thereby blocking the expression or initiating the degradation of the co-receptor nucleic acid. Accordingly, in some embodiments, the inhibitor of an HIV co-receptor is capable of reducing expression of the HIV co-receptor when the expression vector encoding the inhibitor is expressed in a host cell.

In some embodiments, an expression vector of the present disclosure comprises a nucleic acid sequence encoding an antisense oligonucleotide having a sequence that is substantially complementary to at least a portion of a nucleic acid sequence encoding an HIV co-receptor. Examples of HIV co-receptors include CCR5 and/or CXCR4. As used herein, “substantially complementary” refers to a sequence that is at least about 75%, 80%, 85%, 90%, 95%, 96%, 97%, 98%, or 99% complementary to a target polynucleotide sequence. In one embodiment, the antisense oligonucleotide has a sequence that is 100% complementary to at least a portion of a nucleic acid sequence encoding CCR5 or CXCR4. In some embodiments, the antisense oligonucleotide can be from about 15 to about 30 nucleotides in length. In other embodiments, the antisense oligonucleotide can be from about 19 to about 25 nucleotides in length.

In certain embodiments, a nucleic acid sequence of the expression vector encodes a shRNA having a stem-loop structure, wherein the stem or double-stranded region comprises a first portion of the double-stranded region having a sequence that is identical to part of the sequence of the HIV co-receptor, and wherein a second portion of the double-stranded region comprises a sequence that is complementary to another part of the sequence of the HIV co-receptor. In some embodiments, the loop region of the shRNA can comprise from about 2 to about 19 nucleotides. In one particular embodiment, the first nucleic acid sequence encodes a shRNA comprising the sequence of SEQ ID NO: 25. In another particular embodiment, the shRNA loop structure comprises the sequence of SEQ ID NO:43.

Nucleic Acid Sequences which Encode a Protein that Inhibits HIV Fusion

In some embodiments, the expression vectors of the present disclosure comprise a nucleic acid sequence encoding a protein that inhibits HIV fusion to a target cell. In some embodiments, the protein that inhibits HIV fusion to a target cell is a C46 protein. C46 is a membrane anchored fusion inhibitor derived from the C-terminal heptad repeat of HIV gp41 fused with a human immunoglobulin hinge region and a CD34 transmembrane domain. C46 is a potent HIV fusion inhibitor, which the skilled artisan will appreciate that, in a sense, is analogous to the FDA approved soluble drug enfuvirtide (T20). While analogous, the C46 protein is not T20 (e.g. the C46 peptide is about 10 amino acids longer in length than T20) (see, for example, Petit et al. Human Gene Therapy Methods. August 2014, 25(4): 232-240. doi:10.1089/hgtb.2014.034, and Felix et al. Mutations in gp120 Contribute to the Resistance of Human Immunodeficiency Virus Type 1 to Membrane-Anchored C-Peptide maC46, J Virol. 2009 May; 83(10): 4844-4853, the disclosures of which is hereby incorporated by reference herein in its entirety). The skilled artisan will also appreciate that the nucleic acid sequence which encodes a protein that inhibits HIV fusion (e.g. C46 or T20) acts at a point in the HIV life cycle distinct from CCR5 or CXCR4 co-receptor attachment. In some embodiments, the safety of C46 was tested in a phase I clinical trial in which patients received an infusion of autologous T-cells transduced with C46 retroviral vector. Without wishing to be bound by any particular theory, that study indicated that the patients had no gene therapy related adverse effects and did not develop apparent anti-C46 immune reactions.

In some embodiments, the expression vectors of the present disclosure comprise a nucleic acid sequence encoding a protein that inhibits HIV fusion to a target cell other than T20, i.e. the nucleic acid encoding an HIV fusion inhibitor is not T20.

In one embodiment, a nucleic acid sequence of an expression vector encodes a C46 protein comprising the sequence of SEQ ID NO: 26.

In another embodiment, a nucleic acid sequence of an expression vector comprises the sequence of SEQ ID NO: 27.

The skilled artisan will appreciate that other suitable proteins that inhibit HIV fusion to a target cell and can be encoded by a nucleic acid sequence in the expression vectors of the present disclosure and include, without limitation, T20 and its related proteins, enfuvirtide, CP32M, and sifuvirtide (see, e.g., PCT/US2010/036247, the disclosure of which is hereby incorporated by reference herein in its entirety.)

Nucleic Acid Sequences which Encode a Protein that Inhibits HIV Replication

In some embodiments, the expression vectors of the present disclosure comprise a nucleic acid sequence encoding a protein that inhibits HIV replication. In some embodiments, the nucleic acid sequence encodes a tripartite motif-containing 5 alpha (TRIM5α) protein or a derivative or fusion thereof. In other embodiments, the nucleic acid sequence encodes a chimeric TRIM5α. A “chimeric TRIM5α” refers to a TRIM5a protein comprising domains or fragments from TRIM5α proteins from two or more species. In some embodiments, the chimeric TRIM5α comprises an amino terminal domain from a human TRIM5α protein and the carboxy terminal PRYSPRY domain is from a rhesus TRIM5α protein.

In some embodiments, the nucleic acid sequence encodes a TRIM5-cyclophilin fusion protein. In some embodiments, the TRIM5-cyclophilin fusion protein comprises amino acids 1 to about 309 of human TRIM5α fused directly to about full-length human cyclophilin A. In other embodiments, the TRIM5-cyclophilin fusion protein comprises amino acids 1 to about 322 of human TRIM5α fused directly to about full-length human cyclophilin A. In yet other embodiments, the TRIM5-cyclophilin fusion protein comprises amino acids 1 to about 331 of human TRIM5α fused directly to about full-length human cyclophilin A. Further suitable proteins that inhibit HIV replication that can be encoded by the second nucleic acid sequence include, but are not limited to, cyclophilin, E3 ubiquitin, APOBEC3G, and bone marrow stromal cell antigen 2 (BST-2). In some embodiments, the expression vector comprises a nucleic acid sequence which encodes a human TRIM5α protein as provided by SEQ ID NO:28. In other embodiments, the expression vector comprises a nucleic acid sequence having the sequence of SEQ ID NO:29.

Examples of Expression Vectors

In some embodiments, the present disclosure provides an expression vector comprising a first nucleic acid sequence encoding an inhibitor of an HIV co-receptor, a second nucleic acid sequence encoding a protein that inhibits HIV fusion to the target cell, and a third nucleic acid encodes a silencing nucleic acid which targets a sequence of the 5′ LTR of HIV-1to inhibit HIV-1 gene transcription, wherein the first nucleic acid sequence is operably linked to a first promoter, the second nucleic acid sequence is operably linked to a second promoter, and the third nucleic acid is operably linked to a third promoter. In some embodiments, the third nucleic acid targets one of a sequence from about position 143 to about position 161 of the 5′ LTR of HIV-1, a sequence from about position 136 to about position 154 of the 5′ LTR of HIV-1, a sequence from about position 205 to about position 223 of the 5′ LTR of HIV-1, or a sequence from about position 350 to about position 368 of the 5′ LTR of HIV-1 to inhibit HIV-1gene transcription. In some embodiments, the first and second nucleic acid sequences are transcribed from a single promoter. In other embodiments, two of the three nucleic acid sequences are transcribed from a single promoter. In some embodiments, the second nucleic acid sequence encoding a protein that inhibits HIV fusion to the target cell is selected from a protein other than T20, i.e. the protein is not T20. In some embodiments, the protein other than T20 is C46.

In other embodiments, the present disclosure provides an expression vector comprising a first nucleic acid sequence which encodes an siRNA or shRNA molecule which comprises a double-stranded region having a sequence that is substantially identical and complementary to CCR5; a second nucleic acid sequence which encodes a C46 protein; and a third nucleic acid sequence which encodes a silencing nucleic acid which targets a sequence of the 5′ LTR of HIV to inhibit HIV gene transcription; wherein the first nucleic acid sequence is operably linked to a first promoter, the second nucleic acid sequence is operably linked to a second promoter, and the third nucleic acid is operably linked to a third promoter. In some embodiments, the third nucleic acid targets one of a sequence from about position 143 to about position 161 of the 5′ LTR of HIV-1, a sequence from about position 136 to about position 154 of the 5′ LTR of HIV-1, a sequence from about position 205 to about position 223 of the 5′ LTR of HIV-1, or a sequence from about position 350 to about position 368 of the 5′ LTR of HIV-1 to inhibit HIV-1 gene transcription. In some embodiments, the first and second nucleic acid sequences are transcribed from a single promoter. In other embodiments, two of the three nucleic acid sequences are transcribed from a single promoter. In some embodiments, the expression vector comprises a fourth nucleic acid sequence, such as one encoding another transcriptional gene silencing element.

In yet other embodiments, the present disclosure provides an expression vector which comprises a first, second, third and fourth nucleic acid sequence, wherein the first nucleic acid sequence encodes an inhibitor of an HIV co-receptor (e.g., shRNA to CCR5 or CXCR4), the second nucleic acid sequence encodes a fusion inhibitor (e.g., C46), the third nucleic acid sequence encodes an inhibitor of HIV replication (e.g., TRIM5α protein or a derivative or fusion thereof), and the fourth nucleic acid sequence encodes a silencing nucleic acid which targets one of a sequence from about position 143 to about position 161 of the 5′ LTR of HIV-1, a sequence from about position 136 to about position 154 of the 5′ LTR of HIV-1, a sequence from about position 205 to about position 223 of the 5′ LTR of HIV-1, or a sequence from about position 350 to about position 368 of the 5′ LTR of HIV-1 to inhibit HIV-1 gene transcription. In some embodiments of the disclosure, the inhibitor of HIV-1 gene transcription, the inhibitor of an HIV co-receptor and the inhibitor of HIV fusion to the target cell or inhibitor of HIV replication are expressed from different promoters on the expression vector. In one embodiment, the inhibitor of an HIV co-receptor (e.g. CCR5 or CXCR4) is expressed from a RNA polymerase III promoter, while the inhibitor of HIV fusion and/or replication is expressed from a RNA polymerase II promoter. The person of ordinary skill in the art will appreciate that two different inhibitors can be expressed in different ratios from the expression construct.

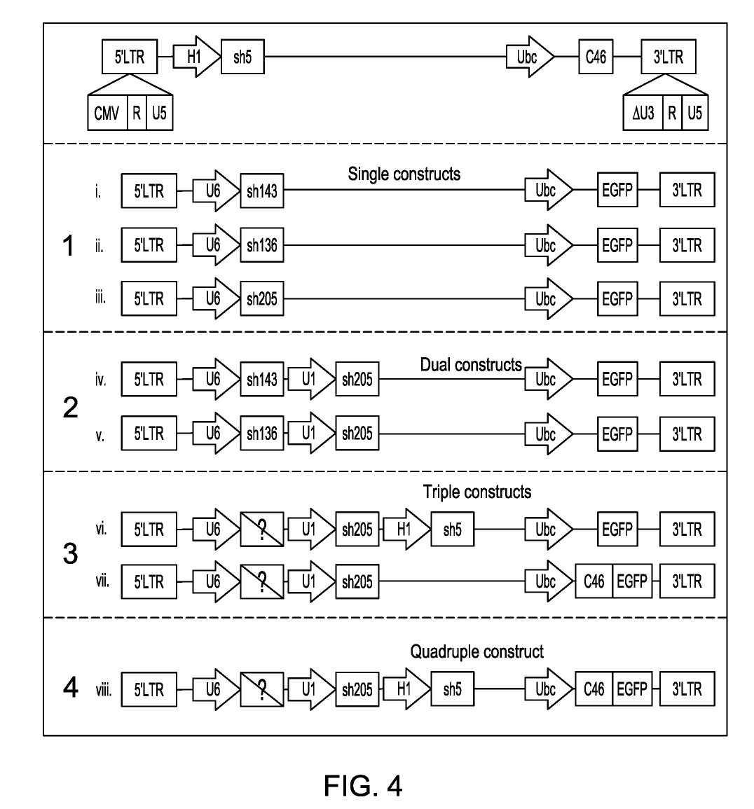

Other examples of expression vectors of the present disclosure are illustrated in FIG. 4. FIG. 4 also illustrates specific promoters to which each nucleic acid of the expression vector is operably linked.

Compositions and Pharmaceutical Compositions

The present disclosure also provides for compositions, including pharmaceutical compositions, comprising one or more expression vectors as disclosed herein. By way of example, a composition may comprise an expression vector comprising a first nucleic acid comprising a transcriptional gene silencing element which targets a sequence within the 5′ LTR of HIV, a second nucleic acid encoding a protein that inhibits HIV fusion to a target cell or HIV replication, and a third nucleic acid encoding an inhibitor of an HIV co-receptor. By way of another example, in some embodiments, a composition comprises an expression vector having a first nucleic acid sequence encoding a shRNA targeting CCR5 (or CXCR4), a second nucleic acid sequence encoding a C46 protein, and a third nucleic acid, namely a silencing nucleic acid, which targets a sequence of the 5′ LTR of HIV.

In some embodiments, the present disclosure also provides for compositions comprising (i) an expression vector or a composition as disclosed in U.S. Publication No. 2012/0201794 (the disclosure of which is incorporated herein by reference in its entirety); and (ii) at least one transcriptional gene silencing element which targets a sequence within the 5′ LTR of HIV, as disclosed herein. For example, in some embodiments, such a composition may comprise (i) an expression vector comprising at least two nucleic acid sequences selected from the group consisting of a nucleic acid sequence that encodes an inhibitor of an HIV co-receptor; a nucleic acid sequence that encodes a protein that inhibits HIV fusion to a target cell; a nucleic acid sequence that encodes a protein that inhibits HIV replication; and a nucleic acid sequence that encodes an inhibitor of viral entry; and (ii) at least one transcriptional gene silencing element or an expression vector, cell, or composition comprising at least one transcriptional gene silencing element. By way of example, such a composition may comprise (i) an expression vector having a first nucleic acid sequence encoding a shRNA targeting CCR5 (or CXCR4), and a second nucleic acid sequence encoding a C46 protein; and (ii) a silencing nucleic acid which targets a sequence of the 5′ LTR of HIV.

In some embodiments, pharmaceutical compositions comprise an effective amount of at least one of the expression vectors or compositions as described herein and a pharmaceutically acceptable carrier. For instance, in certain embodiments, the pharmaceutical composition comprises an effective amount of an expression vector and a pharmaceutically acceptable carrier. As used herein, an “effective amount” is an amount sufficient to cause inhibition of HIV gene expression. An affective amount can be readily determined by those skilled in the art based on factors such as body size, body weight, age, health, sex of the subject, ethnicity, and viral titers.

Any of the expression vectors disclosed herein (or combinations thereof) may be formulated as a pharmaceutical composition. The phrases “pharmaceutically acceptable” or “pharmacologically acceptable” refer to molecular entities and compositions that do not produce adverse, allergic, or other untoward reactions when administered to an animal or a human. For example, the silencing nucleic acid may be formulated with a pharmaceutically acceptable carrier. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. Methods for the formulation of compounds with pharmaceutical carriers are known in the art and are described in, for example, in Remington's Pharmaceutical Science, (17th ed. Mack Publishing Company, Easton, Pa. 1985); and Goodman & Gillman's: The Pharmacological Basis of Therapeutics (11th Edition, McGraw-Hill Professional, 2005); the disclosures of each of which are hereby incorporated herein by reference in their entirety.

The pharmaceutical compositions may comprise any of the expression vectors or compositions disclosed herein (or combinations thereof) in any concentration that allows the silencing nucleic acid administered to achieve a concentration in the range of from about 0.1 mg/kg to about 1 mg/kg. The pharmaceutical compositions may comprise the expression vector (or combinations thereof) in an amount of from about 0.1% to about 99.9% by weight. Pharmaceutically acceptable carriers suitable for inclusion within any pharmaceutical composition include water, buffered water, saline solutions such as, for example, normal saline or balanced saline solutions such as Hank's or Earle's balanced solutions), glycine, hyaluronic acid etc. The pharmaceutical composition may be formulated for parenteral administration, such as intravenous, intramuscular or subcutaneous administration. Pharmaceutical compositions for parenteral administration may comprise pharmaceutically acceptable sterile aqueous or non-aqueous solutions, dispersions, suspensions or emulsions as well as sterile powders for reconstitution into sterile injectable solutions or dispersions. Examples of suitable aqueous and non-aqueous carriers, solvents, diluents or vehicles include water, ethanol, polyols (such as glycerol, propylene glycol, polyethylene glycol, etc.), carboxymethylcellulose and mixtures thereof, vegetable oils (such as olive oil), injectable organic esters (e.g. ethyl oleate).

The pharmaceutical compositions may comprise any of the expression vectors disclosed herein (or combinations thereof) in an encapsulated form. For example, the expression vectors may be encapsulated by biodegradable polymers such as polylactide-polyglycolide, poly(orthoesters) and poly(anhydrides), or may be encapsulated in liposomes or microemulsion. Liposomes may be, for example, lipofectin or lipofectamine. Another example may comprise the expression vectors disclosed herein (or combinations thereof) in or on anucleated bacterial minicells (Giacalone et al, Cell Microbiology 2006, 8(10): 1624-33). The expression vectors disclosed herein (or combinations thereof) may be combined with nanoparticles.

The pharmaceutical composition may be formulated for oral administration. Solid dosage forms for oral administration may include, for example, tablets, dragees, capsules, pills, and granules. In such solid dosage forms, the composition may comprise at least one pharmaceutically acceptable carrier such as sodium citrate and/or dicalcium phosphate and/or fillers or extenders such as starches, lactose, sucrose, glucose, mannitol, and silicic acid; binders such as carboxylmethylcellulose, alginates, gelatin, polyvinylpyrrolidone, sucrose and acacia; humectants such as glycerol; disintegrating agents such as agar-agar, calcium carbonate, potato or tapioca starch, alginic acid, silicates, and sodium carbonate; wetting agents such as acetyl alcohol, glycerol monostearate; absorbants such as kaolin and bentonite clay; and/or lubricants such as talc, calcium stearate, magnesium stearate, solid polyethylene glycol, sodium lauryl sulfate, and mixtures thereof. Liquid dosage forms for oral administration may include, for example, pharmaceutically acceptable emulsions, solutions, suspensions, syrups and elixirs. Liquid dosages may include inert diluents such as water or other solvents, solubilizing agents and/or emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (such as, for example, cottonseed oil, corn oil, germ oil, castor oil, olive oil, sesame oil), glycerol, tetrahydrofurfuryl alcohol, polyethylene glycols and fatty acid esters of sorbitan, and mixtures thereof.

The pharmaceutical compositions may comprise penetration enhancers to enhance their delivery. Penetration enhancers may include fatty acids such as oleic acid, lauric acid, capric acid, myristic acid, palmitic acid, stearic acid, linoleic acid, linolenic acid, dicaprate, reclineate, monoolein, dilaurin, caprylic acid, arachidonic acid, glyceryl 1-monocaprate, mono and di-glycerides and physiologically acceptable salts thereof. The compositions may further include chelating agents such as, for example, ethylenediaminetetraacetic acid (EDTA), citric acid, salicylates (e.g. sodium salycilate, 5-methoxysalicylate, homovanilate). The expression vectors disclosed herein (or combinations thereof) may be delivered combined with minicells or nanoparticles.

In some embodiments, the compositions may be formulated as nanocapsules. Nanocapsule structure consists of nano-vesicular system that is formed in a core-shell arrangement. The shell of a typical nanocapsule is made of a polymeric membrane or coating. In some embodiments, the nanocapsules are derived from a biodegradable or bioerodable polymeric material.

In some embodiments, the nanocapsule is an enzymatically degradable nanocapsule. In some embodiments, the nanocapsule consists of a single-protein core and a thin polymeric shell cross-linked by peptides. In some embodiments, a nanocapsule may be selected such that it is specifically recognizable and able to be cleaved by a protease. In some embodiments, the cleavable cross-linkers include a peptide sequence or structure that is a substrate of a protease or another enzyme. Suitable nanocapsules, methods of synthesis, and/or methods of encapsulation, are further disclosed in United States Patent Publication No. 2011/0274682, the disclosure of which is hereby incorporated by reference herein in its entirety.

In other embodiments, the compositions may be formulated as bio-nanocapsules, which are nano-size capsules produced by a genetically engineered microorganism. It is possible to use, as a bio-nanocapsule, a virus protein-derived or modified virus protein-derived particle, such as a virus surface antigen particle (e.g., a hepatitis B virus surface antigen (HBsAg) particle). It is also possible to use, as a bio-nanocapsule, a nano-size capsule comprising a lipid bilayer membrane and a virus protein-derived or modified virus protein-derived particle such as a virus surface antigen particle (e.g., a hepatitis B virus surface antigen (HBsAg) particle). Such particles can be purified from eukaryotic cells such as yeasts, insect cells, and mammalian cells. The size of a capsule that can be used is approximately 10 nm to 500 nm, preferably 20 nm to 250 nm, and most preferably 80 nm to 150 nm can be used.

Methods of Treatment

In general, the present disclosure provides various methods including, (i) methods of inhibiting HIV transcription; (ii) methods of inhibiting HIV replication; (iii) methods of treating an HIV infection; (iv) methods of preventing an HIV infection; (v) methods of reducing an HIV infection; (vi) methods of preventing or reducing a productive HIV infection in a subject not suffering from any HIV infection; (vii) methods of inhibiting binding an HIV co-receptor; and (viii) methods of inhibiting fusion of HIV to a target cell. These methods are carried out or achieved by administering, or contacting a cell with, an expression vector as described herein, a composition comprising an expression vector and/or other component or active agents, or co-administering (a) composition comprising an expression vector, and (b) another component or active agent. The present disclosure also provides for the use of the present expression vectors, compositions comprising expression vectors, etc. to inhibit HIV gene transcription, binding to HIV co-receptor, fusion of HIV to the target cell or HIV replication in the manufacture of a medicament for treating or preventing HIV infection in a subject.

More specifically, the present disclosure provides a method of treating, preventing, and/or reducing HIV infection in a patient or subject, such as those in need to treatment or therapy. As used herein, “treating” means affecting a subject, tissue or cell to obtain a desired pharmacological and/or physiological effect and includes inhibiting the condition, i.e. arresting its development; or relieving, mitigating or ameliorating the effects of the condition i.e. cause reversal or regression of the effects of the condition. “Reducing” means to lower the degree or intensity of a condition from occurring in a cell or a subject that may be at risk of having the condition. As used herein, “preventing” means preventing a condition from occurring in a cell or subject that may be at risk of having the condition, but does not necessarily mean that condition will not eventually develop, or that a subject will not eventually develop a condition. Preventing includes delaying the onset of a condition in a cell or subject. In one embodiment, treating achieves the result of preventing or reducing HIV replication. Treating may also achieve the result of preventing reactivation of latent HIV1-virus in the recipient subject.

In some embodiments, preventing and/or reducing HIV infection in a subject comprises preventing and/or reducing HIV infection in a subject suffering from HIV infection. As used herein, the expression “preventing or reducing HIV infection in a subject suffering from HIV infection” refers to eliminating, reducing or delaying the production of infectious HIV virus in a subject already infected with HIV such that infection of uninfected tissue with HIV in the subject is prevented, reduced or delayed. In another embodiment, preventing or reducing HIV infection in a subject comprises preventing or reducing a productive HIV infection in a subject not suffering from HIV infection. As used herein, “preventing or reducing a productive HIV infection in a subject not suffering from HIV infection” refers to preventing or reducing development of an HIV infection of a subject who has not been previously infected with HIV.

As used herein, the term “subject” refers to a mammal that is susceptible to HIV infection, such as a human, mouse or primate. Typically, the mammal is a human (homo sapiens).

As used herein, the term “administering” means providing a composition, formulation, or specific agent to a subject in need of treatment, including those described herein.

It will be understood that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In some embodiments, the methods disclosed herein comprise administering to the subject an effective amount of an expression vector or pharmaceutical composition comprising an expression vector as provided herein. Administration of such compositions can confer resistance to infection by R5 and X4 tropic strains of HIV. In some embodiments, the subject is human. The subject may be HIV negative or HIV positive. In some embodiments, the subject may be naive to cART therapy, receiving cART therapy, failing or failed on cART therapy. In other embodiments, the subject may have progressed to AIDS or have an AIDS-defining illness (e.g., AIDS/lymphoma).

By way of example, in some embodiments, the methods comprise administering to the subject a pharmaceutical composition comprising an expression vector, wherein the expression vector comprises a first nucleic acid sequence encoding a shRNA targeting CCR5 (or CXCR4), a second nucleic acid sequence encoding a C46 protein, and a third nucleic acid sequence encoding a silencing nucleic acid which targets a sequence in the 5′ LTR of HIV.

In some embodiments is a method of treating an HIV infection in a subject comprising administering to the subject an effective amount of an expression vector or a composition comprising an expression vector, wherein the expression vector comprises (i) at least one nucleic acid sequence encoding a transcriptional gene silencing element; and (ii) at least two other nucleic acid sequences selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry. In other embodiments is a method of preventing and/or reducing an HIV infection in a subject comprising administering to the subject an effective amount of an expression vector or a composition comprising an expression vector, wherein the expression vector comprises (i) at least one nucleic acid sequence encoding a transcriptional gene silencing element; and (ii) at least two other nucleic acid sequences selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry.

In other embodiments is a method of treating, preventing, and/or reducing an HIV infection in a subject comprising administering to the subject an effective amount of an expression vector or a composition comprising an expression vector, wherein the expression vector comprises a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. In some embodiments, the nucleic acid encoding an HIV fusion inhibitor is other than T20.

In other embodiments is a method of treating, preventing, and/or reducing HIV infection in a subject, comprising administering to the subject an effective amount of a composition comprising (i) an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) a nucleic acid that encodes at least one transcriptional gene silencing element which targets a sequence of the 5′ LTR of HIV.

In yet other embodiments, the methods of treating an HIV infection in a subject comprising co-administering (i) an effective amount of an expression vector, a pharmaceutical composition comprising an expression vector, or a cell comprising at least two nucleic acids, wherein the at least two nucleic acids are selected from the group consisting of a nucleic acid sequence that encodes an inhibitor of an HIV co-receptor; a nucleic acid sequence that encodes a protein that inhibits HIV fusion to a target cell; a nucleic acid sequence that encodes a protein that inhibits HIV replication; and a nucleic acid sequence that encodes an inhibitor of viral entry; and (ii) an effective amount of at least one silencing nucleic acid which targets a sequence in the 5′ LTR of HIV. In some embodiments, the co-administering is simultaneous. In other embodiments, the expression vector and the at least one transcriptional gene silencing element are administered at different times.

In yet further embodiments, the methods of treating, preventing, and/or reducing HIV infection in a subject comprising co-administering (i) an effective amount of an expression vector, a pharmaceutical composition comprising an expression vector, or a cell as disclosed in U.S. Publication No. 2012/0201794 (wherein the expression vectors from the '794 Publication are referred to as “Cal-1” herein); and (ii) an effective amount of at least one transcriptional gene silencing element, a composition or conjugate comprising at least one transcriptional gene silencing element, or a cell comprising at least one transcriptional gene silencing element. In some embodiments, the transcriptional gene silencing element is selected from those described in PCT/AU2015/050507, filed Aug. 28, 2015, the disclosure of which is hereby incorporated by reference herein in its entirety. In some embodiments, the co-administering is simultaneous. In other embodiments, the expression vector and the at least one transcriptional gene silencing element are administered at different times. The skilled artisan will be able to provide an appropriate dosing regime and/or schedule for co-administration of the expression vector and the transcriptional gene silencing element.

In yet further embodiments is a method of treating and/or preventing HIV infection in a subject comprising transducing hematopoietic cells with the expression vector, and transplanting the transduced hematopoietic cells in the subject, wherein the transduced hematopoietic cells are resistant to HIV infection, wherein the expression vector comprises a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1. In some embodiments, the method comprises transducing hematopoietic cells (e.g., HPSC, CD4+ T lymphocytes, CD8+ T lymphocytes, or monocyte/macrophages) with an expression vector of the disclsoure and transplanting the transduced cells in the patient, wherein the transduced cells are resistant to HIV infection. In some embodiments, the hematopoietic cells are hematopoietic progenitor/stem cells (HPSC) that generate granulocytes, monocyte/macrophages, and lymphocytes that are resistant to HIV infection following transplantation into a patient. In some embodiments, the HPSC are autologous and CD34 positive. In some embodiments, the transduced HPSC can generate granulocytes, monocyte/macrophages, and lymphocytes that are resistant to infection by R5 and X4 tropic strains of HIV. In some embodiments, the transduced HPSC can generate granulocytes, monocyte/macrophages, and lymphocytes that are resistant to infection by HIV strains that are resistant to cART. Alternatively, the present disclosure also encompasses the use of such cells in the manufacture of a medicament for treating and/or preventing an HIV infection in a subject.

A method for treating a human host patient infected with HIV, comprising the steps of extracting hematopoietic stem cells from the circulating blood or bone marrow of the host patient; treating hematopoietic stem cells with an expression vector (or a composition comprising an expression) as disclosed herein; and re-administering or transplanting the treated hematopoietic stem cells into the same patient. In some embodiments, the expression vector comprises a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence of a 5′ LTR of HIV selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1.

Host Cells and Methods of Making Host Cells

The present disclosure also provides a host cell comprising the novel expression vectors of the present disclosure. A “host cell” or “target cell” means a cell that is to be transformed using the methods and expression vectors of the disclosure. In some embodiments, the host cells are mammalian cells in which the expression vector can be expressed. Suitable mammalian host cells include, but are not limited to, human cells, murine cells, non-human primate cells (e.g. rhesus monkey cells), human progenitor cells or stem cells, 293 cells, HeLa cells, D17 cells, MDCK cells, BHK cells, and Cf2Th cells. In certain embodiments, the host cell comprising an expression vector of the disclosure is a hematopoietic cell, such as hematopoietic progenitor/stem cell (e.g. CD34-positive hematopoietic progenitor/stem cell (HPSC)), a monocyte, a macrophage, a peripheral blood mononuclear cell, a CD4+ T lymphocyte, a CD8+ T lymphocyte, or a dendritic cell. In some embodiments, the host cell is a CCR5+ hematopoietic cell. In other embodiments, the host cell may be a host cell from a patient or matched to a patient. In certain embodiments, a host cell transduced with the expression vectors of the disclosure are resistant to infection by X4 or R5-tropic HIV strains, including HAART resistant strains.

The hematopoietic cells (e.g. HPSC, CD4+ T lymphocytes, CD8+ T lymphocytes, and/or monocyte/macrophages) to be transduced with an expression vector of the disclosure can be allogeneic or autologous. The HPSC are, in some embodiments, CD34-positive and can be isolated from the patient's bone marrow or peripheral blood. The isolated CD34-positive HPSC (and/or other hematopoietic cell described herein) is, in some embodiments, transduced with an expression vector as described herein. For example, in one embodiment the expression vector comprises a first nucleic acid sequence encoding an inhibitor of an HIV co-receptor, a second nucleic acid sequence encoding a protein that inhibits HIV fusion to a target cell, and a third nucleic acid sequence that encodes a transcriptional gene silencing element which targets a sequence within the 5′ LTR of HIV.

Following transduction of the hematopoietic cells (e.g., FIPSC, CD4+ T lymphocytes, CD8+ lymphocytes, or monocyte/macrophages) with an expression vector as disclosed herein, the transduced cells are reintroduced or transplanted back into the patient. The transduced cells can be injected parenterally into the patient, or reintroduced by any other route known in the art. In some embodiments, the transduced hematopoietic cells are injected intravenously into the patient. In some embodiments, the hematopoietic cell is a HPSC and an effective dose is an amount that is sufficient to at least partially reconstitute the immune system with HIV-resistant cells. The dose could be administered in one or more administrations and may be from about 0.5×106 HPSC per kg patient weight to about 1×109 HPSC per kg patient weight. In another embodiment, the hematopoietic cell is a CD4+ T lymphocyte, a CD8+ T lymphocyte, or a monocyte/macrophage and an effective dose may be from about 1×109 cells per patient to 1×1011 cells per patient. However, the precise determination of what would be considered an effective dose may be based on factors individual to each patient, including their size, age, severity of HIV infection (e.g. viral titer), and amount of time since contraction of the virus. One skilled in the art, specifically a physician, would be able to determine the number of transduced hematopoietic cells which would constitute an effective dose without being subjected to undue experimentation.

Kits

In some embodiments is a kit comprising an expression vector or a composition comprising an expression as described herein. The kit may comprise a container, where the container may be a bottle comprising the expression vector or composition in oral or parenteral dosage form, each dosage form comprising a unit dose of the expression vector or composition comprising the expression vector. The kit may comprise a label or the like, indicating treatment of a subject according to the present method.

Detailed Methodology

Synthesis

The present disclosure also includes a method of producing a viral expression vector that is capable of inhibiting binding of HIV to the cell and preventing HIV fusion to the cell and/or HIV replication when expressed in a host cell. In one embodiment, the method comprises synthesizing a cDNA of a gene which expresses a protein capable of preventing HIV fusion into a cell or HIV replication; cloning the synthesized cDNA into a restriction site in a viral vector; and inserting an expression unit capable of down-regulating expression of an HIV co-receptor into a restriction site in the vector. The cDNA can be from any gene which expresses any of the protein fusion or replication inhibitors described herein. In some embodiments, the cDNA is a C46 cDNA. In another embodiment, the cDNA is a TRIM5α cDNA or a cyclophilin fusion thereof. The expression unit capable of down-regulating expression of a HIV co-receptor can be any of the inhibitory RNA molecules described herein, such as siRNA, shRNA, or antisense targeting the co-receptor. In one embodiment, the expression unit is a shRNA targeting CCR5.

In some embodiments, an shRNA targeting an HIV-1 promoter is cloned into an existing vector, such as a vector expressing a shRNA targeting CCR5 and/or C46. In some embodiments, the existing plasmid is cut with a restriction enzyme. An in-fusion enzyme may then be used to fuse four or more sets of DNA segments by homologous recombination, the four or more sets of DNA sections engineered to contain approximately 15 bp of homologous sequence, allowing for recombination of individual G Blocks. A first G Block, namely G Block 1, contains a U1 promoter sequence and half of the shPromA sequence. A second G Block, namely G Block 2, contains the other half of the shPromA sequence. Recombination is followed by transformation of E.coli with antibiotic selection.

RNA duplexes. Double-stranded RNA duplexes were designed to target the HIV-1 5′LTR region. RNA duplexes were synthesized by Invitrogen. Each siRNA construct was 19 bp in length and was designed with a 3′TT overhang.

Cell culture. Media and reagents for cell culture were purchased from Gibco. HeLa T4+ cells were grown in Dulbecco's modified Eagle's medium (DMEM) containing 10% fetal calf serum, 5 U/ml penicillin and 50 mg/mL streptomycin (supplemented DMEM) and incubated at 37° C. in a humidified incubator with 5% CO2. HeLa T4+ cells were maintained under selection with G418 (500 μg/mL) and Hygromycin (300 μg/mL), respectively.

Live HIV-1 infection and siRNA transfection. Cell cultures for time course experiments using replication competent virus were seeded with 5×104 HeLa-T4+ cells, incubated overnight and then transfected with 50 pM of the appropriate siRNA panel. The following day HeLa-T4+ siRNA-transfected cultures were infected with HIV-1 strain SF162, using 140 pg/μL. Supernatants were harvested over a time course of 15 days for analysis of virus production using the reverse transcriptase (RT) assay described below. Cultures for the ChIP experiment were seeded with 2×105 HeLa-T4+ cells, incubated overnight and then infected with HIV-1 strain SF162 using 1000 pg/μL and the infection was allowed to proceed for 3 days. The infected cultures were then transfected with 300 pM siRNAs 143, 136, 205, PromA or mock-transfected for 48 h prior to harvest for the ChIP assay. Transfections were performed using RNAiMax (Invitrogen, Life Technologies) according to the manufacturer's instructions.

Viral Quantitation. Reverse transcriptase (RT) activity in culture supernatants was determined as described previously (Suzuki, K., Craddock, B. P., Okamoto, N., Kano, T., Steigbigel, R. T., J Virol Methods, 44: 189-98, 1993). HIV-1 mRNA was quantified using a real-time RT-PCR assay specific for HIV-gag as previously described (Suzuki et al., J RNAi Gene Silencing, 2005). Briefly, RT-PCR reactions were performed with SuperScript One-step RT-PCR (Invitrogen) using 0.4 μM of both sense and anti-sense primers, and 0.1 μM of sequence-specific fluorogenic Taqman probe. Standard curves were constructed using genomic HIV plasmid pNL4-3 for HIV-1 and a TA-cloned PCR fragment of beta-actin (Invitrogen, Mount Waverley, Australia). The primers and probes used are provided by SEQ ID NO: 30, SEQ ID NO: 31, SEQ ID NO: 32, SEQ ID NO: 33, SEQ ID NO: 34, and SEQ ID NO: 35.

Drug Treatments.

J-Lat 9.2 latent cell model cultures were treated with various concentrations of SAHA (0-100 uM), TNF (0-100 ng/mL) or a combination of SAHA/TNF (0-25 uM, 0-100 ng/mL) for 48 h. GFP expression was measured using flow cytometry (LSRFortessa-BD) to detect reactivation of integrated HIV-1 provirus and analyzed using FlowJo.

Chromatin Immunoprecipitation (ChIP) assays. HeLa T4+ cells (5×105) were seeded into T-25 flasks and 24 h later infected with HIV-1 strain SF162 as described above. At day 3 post-infection, the cultures were transfected with 300 pmol of each of siRNAs 143, 143T, 136, 205 and the current lead siRNA PromA, or mock-transfected as a control culture as described above. Following 48 h post-transfection, cultures were harvested for the ChIP assay using the EZ Magna A/G ChIP Kit (Millipore, Australia) following manufacturer's instructions. Pellets were sonicated for 20 minutes (1 min off, 1 min on) in a COVARIS 5 sonicator at 5% Duty cycle: intensity 4, Burst/cycles 200, and protein isolated according to manufacturer's instructions. Immunoprecipitations were performed using 5 ug/ml of each antibody, specific to histones H3K27me3 (#17-622) and H3K9Ac (#17-658) obtained from Merck Millipore, for each 2×106 cell equivalents.

Statistical analysis. ChIP data were tested for significance using a non-parametric Mann-Whitney test and are given as mean±SEM. A p-value of <0.05 was considered statistically significant. All analyses were performed using Graphpad Prism Version 6.0 (Graphpad Software, San Diego, Calif.).

### EXAMPLES

### Example 1

### siRNAs Targeting the U3 Region can Induce HIV-1 Suppression

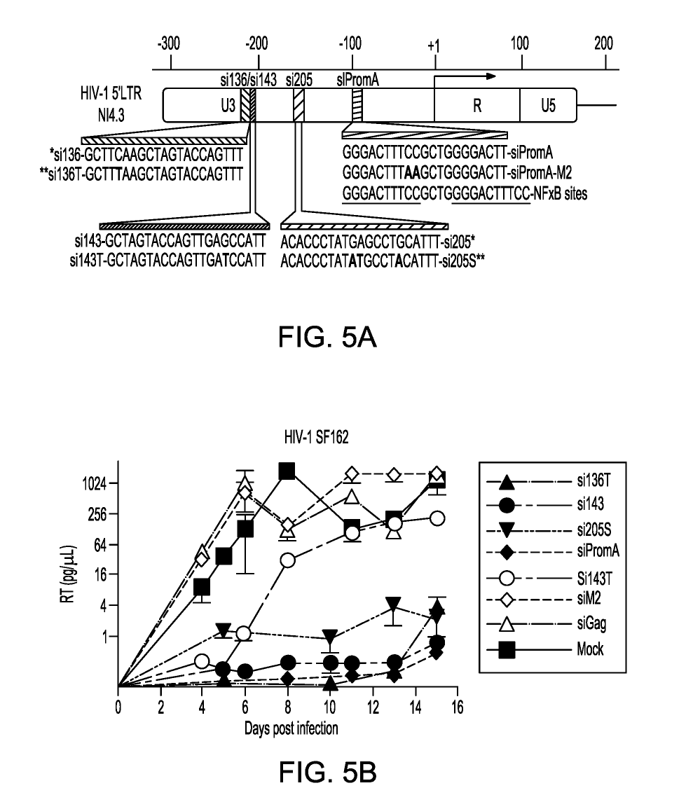

Subtype B strain HIV-1SF162 was used to infect HeLa T4+ cultures and suprenantants were harvested over a prolonged time course and measured for reverse transcriptase (RT). To confirm whether a single mismatch can diminish the suppressive effect, siRNA 143T was included, containing a single T mismatch as it corresponds to a common variation at position 16 across HIV-1 subtypes A, D, F, G and U (FIG. 5) and the HIV-1SF162 3′LTR GenBank sequence (accession number M65024.1). siRNAs, 143, 136T, 205S and PromA, potently suppressed HIV-1SF162 productive infection with >1000-fold reduction in RT activity compared to mock-transfected cells at day 15 post-infection. Interestingly, siRNA 143T suppressed virus infection similarly to siRNA 143 and PromA up to day 6 post-infection, but was unable to maintain virus suppression past this point. These data indicate a single target mismatch is sufficient to disrupt prolonged siRNA-induced HIV-1 virus suppression.

Overall, FIGS. 5A-B show HIV LTR targeted siRNAs potently suppress HIV transcription. To demonstrate the siRNAs targeting the HIV 5′LTR were capable of potently suppressing HIV-1 transcription, HeLa T4+ cells were transfected with the individual siRNAs, infected with HIV-1 SF162 strain and reverse transcriptase (RT) assays were performed over a prolonged time course of infection. The siRNA 143 target sequence is identical to the SF162 strain. To determine whether a single mismatch is critical in reducing the suppressive effect, we also included siRNA 143T, which has a single T mismatch compared to the SF162 strain. This mismatched siRNA 143T corresponds to the SF162 3′LTR sequence available in GenBank (accession number M65024.1). Also included was the sequence specificity control siPromA-M2. Both of the completely matched siRNAs, 143 and PromA, potently suppressed productive infection with HIV-1 strain SF162, with greater than 1000-fold reduction in RT activity compared to mock-transfected cells. Interestingly, 143T suppressed virus infection to a level comparable to 143 and PromA up to day 6 post-infection, but was then unable to maintain virus suppression.

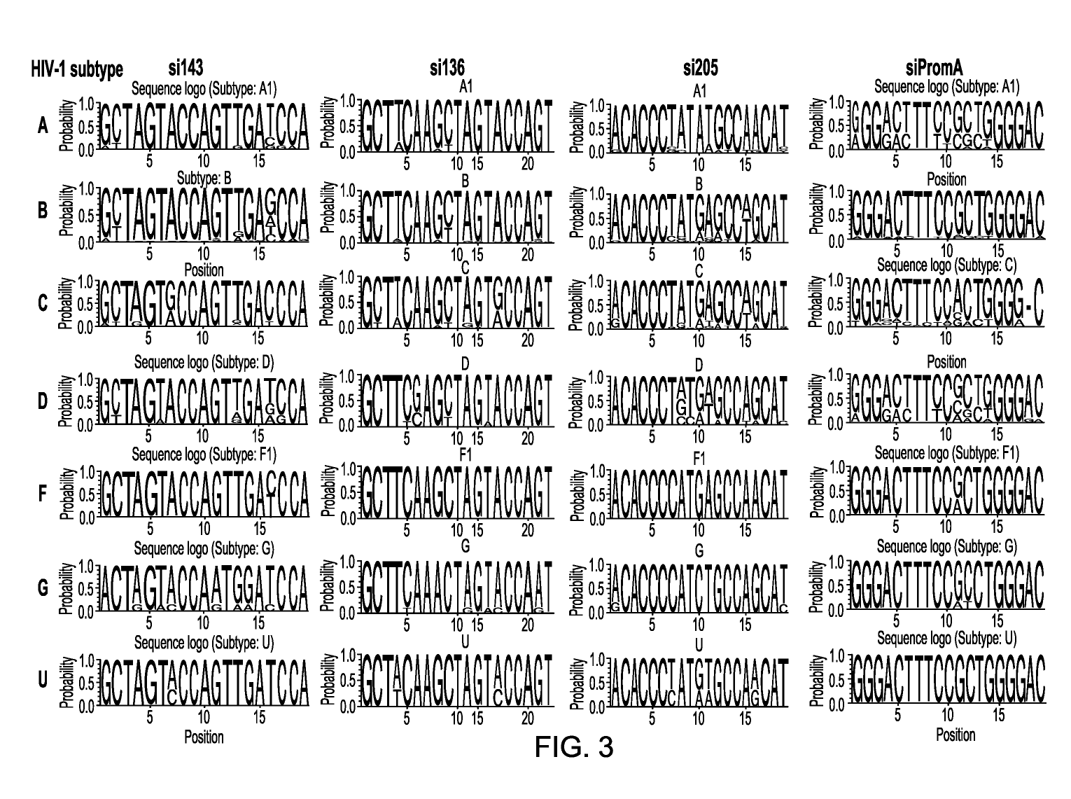

To determine sequence conservation of siRNAs 143, 136 and 205, a sequence alignment across HIV-1 subtypes A through G and U was performed and sequence logos were generated (FIG. 3) (Crooks, Hon, Chandonia, & Brenner, 2004, Genome Res 14: 1188-90; Schneider, & Stephens, 1990, Nucleic Acids Res 18, 6097-100). For example, SiRNA 143 sequence conservation showed 94.9% median identity over 19 nucleotide positions with an identity range of 9/19 nucleotides having >95% conservation, 13/19 nucleotides having >90% conservation and 18/19 nucleotides having >80% conservation. As previously reported siPromA is highly conserved across all subtypes, with the exception of a one bp deletion in subtype C, and showed 98.4% median identity over 19 nucleotide positions (FIG. 3).

### Example 2

### Heterochromatin Markers were Observed in HIV-1 Cultures Suppressed by Novel siRNA Candidate 143 and PromA

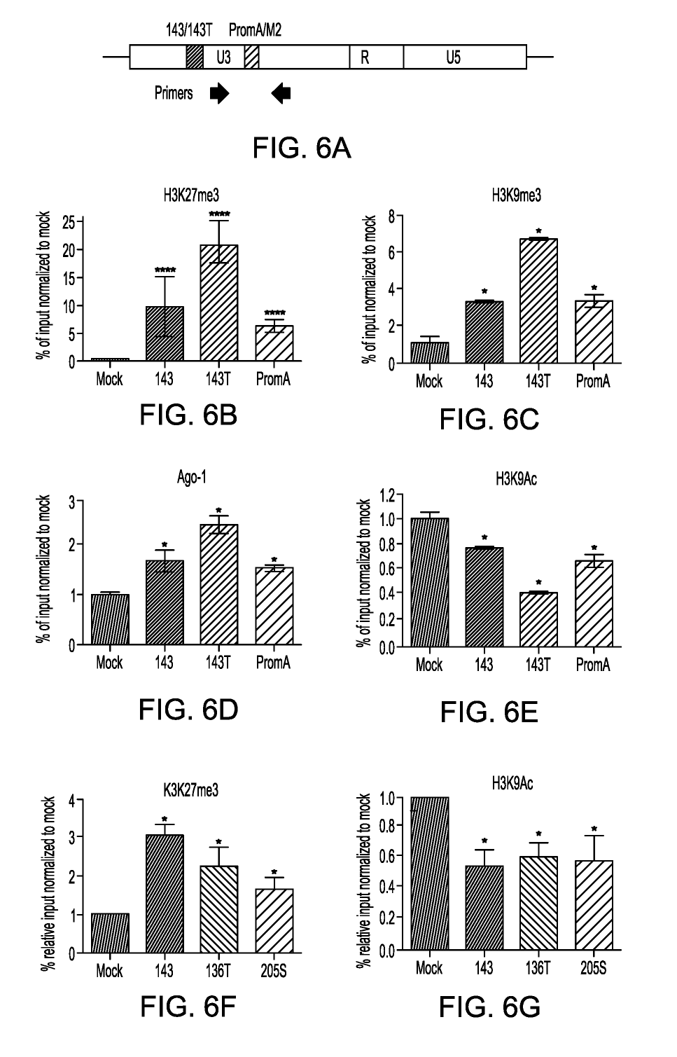

Heterochromatin marks observed in siRNA suppressed HIV-1 cultures as shown in FIGS. 6A-6G. ChIP analysis of heterochromatin marks showed enrichment of H3K27me3 and reduction of H3K9Ac in si143- and siPromA-transfected cells.To determine whether the siRNA 143-mediated HIV-1 suppression was associated with heterochromatin marks as described for PromA, ChIP assays were performed for epigenetic modifications in the 5′LTR 48 h post-transfection. Significant increases were observed in H3K27me3; ˜10 fold in siRNA 143-transfected cultures (p<0.0001) and >5 fold in siRNA PromA-transfected cultures (p<0.0001) compared with the mock-transfected cultures (FIG. 6a). Significant increases were also observed in H3K9me3; ˜5 fold in both siRNA 143- and siRNA PromA-transfected cultures (p<0.001) compared with the mock-transfected cultures (FIG. 6b). Significant recruitment of Ago1 was observed in both siRNA 143- and siPromA-transfected cultures compared to mock-transfected cultures (FIG. 6d). Significant reductions in the histone acetylation marker H3K9Ac were observed in siRNA 143- and PromA-transfected cultures (4-fold, and 6-fold, both p <0.05) (FIG. 6e). Similarly, significant increases in H3K27me3 were reported in siRNA 136 and si205-transfected cultures compared to mock-transfected cultures (FIG. 6f). Finally, significant decreases were reported in siRNA 136- and si205-transfected cultures compared to mock-transfected cultures (FIG. 6g). Together, these data strongly suggest that the siRNA 143, 136 and 205 mimetics all silence HIV through TGS mechanisms.

### Example 3

### shRNA Targeting the 5′LTR Diminish Reactivation Induced by Drug Treatments in Latent HIV-1 J-Lat 9.2 Cells

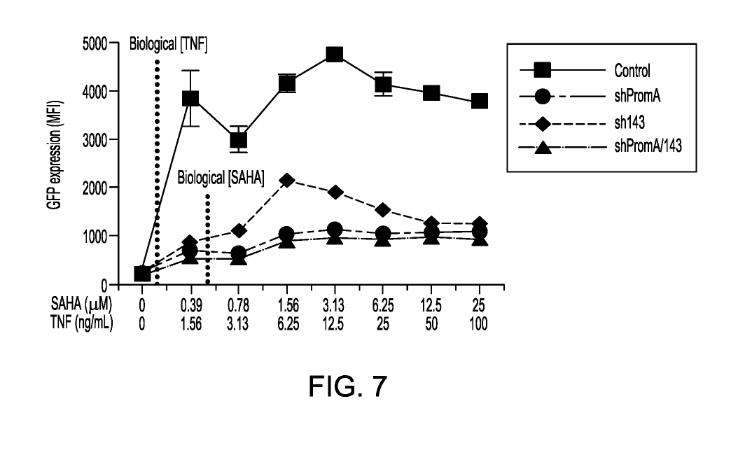

To extend our study and investigate the effect of the siRNA143 target sequence in an HIV-1 latency model, we generated J-Lat 9.2 cells stably transduced with short hairpin sh143, shPromA or transduced with both shPromA and sh143 using separate lentivirus vectors. We then attempted to reactivate integrated latent HIV-1 in J-Lat 9.2 cells using SAHA, TNF or a combination of SAHA/TNF at various concentrations and measured GFP expression as a read out of HIV transcription, at 48 h. J-Lat 9.2 cells transduced with sh143, shPromA or dual sh143/shPromA were observed to be largely resistant to reactivation from SAHA, TNF or combinations of SAHA/TNF at physiological concentrations and showed low level reactivation even at supra-physiological drug concentrations, while those transduced with a shRNA control showed highly elevated GFP expression, indicating reactivation of latent HIV-1 infection (FIG. 7). Importantly, the dual sh143/shPromA transduced J-Lat 9.2 cell line appeared to show a slightly better effect in protecting against reactivation stimuli. These data demonstrate that the HIV-1 latency model J-Lat 9.2 cells are largely resistant to reactivation by drug treatments when transduced with sh143 and/or shPromA.

Robust resistance to reactivation stimuli by shRNA as shown in FIG. 7. J-Lat 9.2 cells transduced with sh143 and shPromA are less susceptible to reactivation by combined TNF/SAHA treatments, particularly at physiological drug concentrations, compared to control cells, as shown by GFP expression which increases upon reactivation.

### Example 4

### Intracellular Viral DNA and mRNA Analysis with Anti-HIV Lentiviral Transgenes—Example of Cal-1 and TGS-Inducing shRNA Lentiviral Constructs

(1) Cal-1 lentiviral vector based on HIV-1 entry inhibitors.

MOLT-4 based in-vitro infectious experiment. Three sets of samples were prepared based on MOLT-4 cells:

a. MOLT-4 cells only without any transduction of Cal-1 lentivirus;

b. A mixture of 80% of MOLT-4 cells without any transduction of Cal-1 and 20% of Cal-1 transduced MOLT-4 cells. Degree of transduced cells was determined by flow analysis of C46 expression. This experimental setting is for monitoring the transgene effect of a mixed population of un-transduced MOLT-4 cells (80%) and Cal-1 transduced MOLT-4 cells (20%);

c. MOLT-4 Cal-1 transduced 100% with Cal-1 lentivirus as determined by C46 expression by Flow cytometry.

About 0.5 million of these cells are infected with HIV-1 BaL and samples were taken at day 4, 7, 10, 14 for analysis intracellular DNA and viral mRNA analysis.

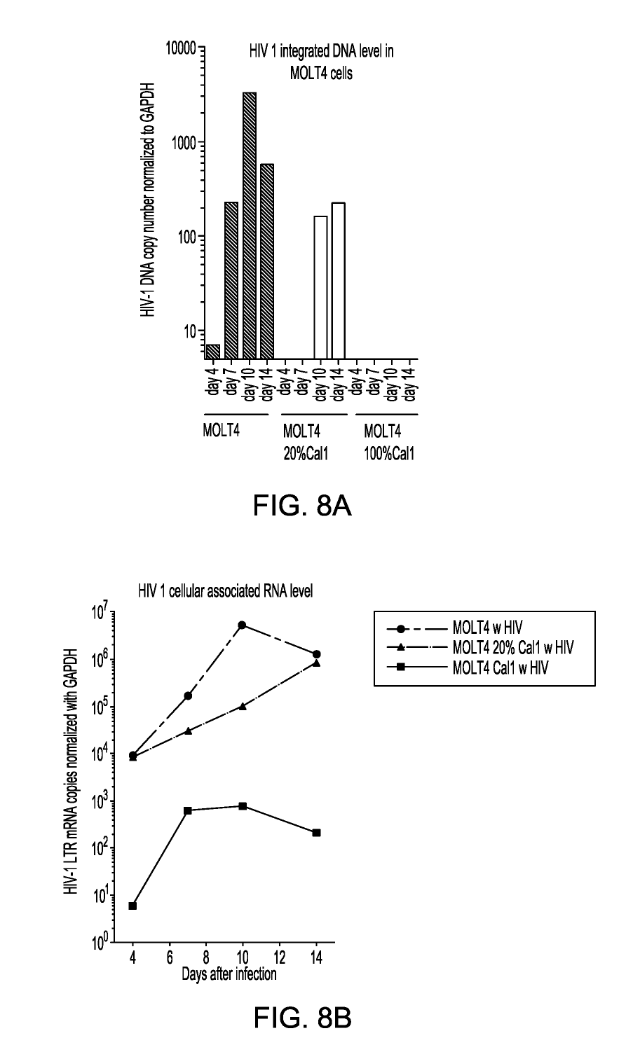

Analysis of intracellular HIV-1 DNA (FIG. 8a): HIV integrated DNA was analyzed using HIV-1 LTR region primer and probe set.

HIV-1 integrated DNA in the MOLT-4 cells was analyzed (FIG. 8a). A significant increase in HIV-1 DNA levels was observed in MOLT-4 cells during the time course. This is indicative of a high level of HIV-1 integration in MOLT-4 cells.

The mixed MOLT-4 cell culture with 20% Cal-1 transduction showed HIV-1 DNA was detected on day 10 and day 14. However, the level of HIV-1 DNA observed was 10-50 fold lower as compared to un-transduced MOLT-4 cells. HIV-1 DNA was not detected in 100% Cal-1 transduced MOLT-4 cells.

In order to detect HIV-1 intercellular RNA level, HIV-1 specific LTR mRNA was analyzed (FIG. 8b). Exceptionally elevated HIV-1 specific cellular associated HIV-1 LTR mRNA was detected in MOLT-4 cells (●) during the culture days.

In contrast, significant reduction of HIV-1 cellular associated HIV-1 LTR mRNA was observed in 100% Cal-1 transduced MOLT-4 cells (▪). These levels were observed to be reduced greater than 1000-fold. The level of reduction in this experimental group (10-100 fold) in HIV-1 cellular associated HIV-1 LTR mRNA in the mixed MOLT-4 cells with 20% transduced efficiency of Cal-1 (▴) is indicative of partial protection from HIV infection. These observations demonstrate the Cal-1 lentivirus vector is able to induce greater than 1000 times reduction of HIV transcription in MOLT-4 cells.

Despite HIV-1 DNA level not being detected in Cal-1 transduced MOLT-4 cells, HIV-1 cellular associated LTR-mRNA was still detectable (▪) in FIG. 8b. This is an indication that a very small level of RNA transcription was taking place from a very small population of HIV-1 integrated DNA in MOLT-4 cells.

An estimated cell number per this DNA PCR was about 1-2×10e5 cell equivalent amount DNA per one PCR reaction. Therefore, HIV-1 detection sensitivity in this assay format is 2 copies per 1-2×10e5 cells. If the HIV-1 DNA integrated frequency were less than 2 copy per 1-2×10e5 cells, the assay would not be capable of detecting HIV-1 DNA in this format.

It is possible that the HIV-1 LTR-mRNA levels observed were the result of a very small fraction of MOLT-4 cells, which failed to be transduced with Cal-1.

(2) TGS-inducing shRNA lentiviral vector.

PM-1 based in-vitro infectious experiment. Three sets of samples were prepared based on the PM-1 CD4+ T-cell line:

a. PM-1 cells without any transduction of shRNA lentivirus;

b. PM-1 cells transduced with shPromA (shRNA targeting HIV-1 promoter region (detail of shPromA construct was described: Suzuki K, et.al, Mol Ther Nucleic Acids 2013, 2:e137);

c. PM-1 cells transduced with shPromA-Scrambled control.

About 0.5 million of these cells were infected with HIV-1 strain JR-FL and samples were taken at day 10 for analysis intracellular DNA and viral mRNA analysis.

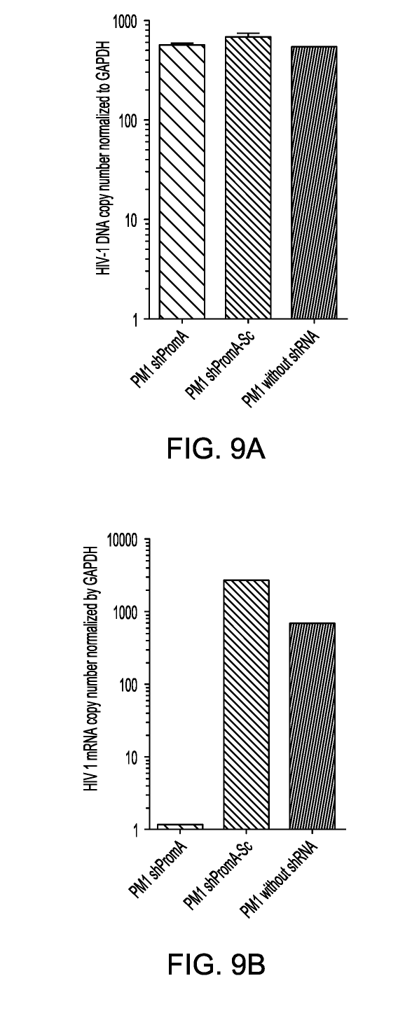

HIV-1 integrated DNA in the PM-1 cells was analyzed (FIG. 9a). The three sets of experimental conditions contained relatively similar amounts of HIV-1 DNA.

In order to detect HIV-1 intercellular RNA level, HIV-1 specific gag mRNA was analyzed (FIG. 9b). Despite the presence of HIV-1 DNA in shPromA transduced PM-1 cells (FIG. 9a), HIV-1 transcription was greatly suppressed. Specifically, around 1000-fold reduction was observed as compared to the un-transduced PM-1 mock infection control and shPromA-Scrambled control. These observations are an indication that shPromA is able to induce around 1000 times reduction of HIV-1 transcription using the PM-1 cell model.

Based on the above data, a combination approach comprising Cal-1 and TSG-inducing shRNA is predicted to generate greater than 106 order of suppression in transduced T-cell lines compared to un-transduced mock infection. Therefore, we expect to observe a complete suppression of expression of HIV-1 transcription by this triple and/or quadruple combination anti-HIV lentivirus vector system.

### Example 5

Applicants believe that triple or quadruple combination vectors according to the present disclosure are at least as safe and efficacious as dual vector constructions, such as Cal-1, described herein. Applicants have shown that autologous transplantation utilizing Cal-1 lentiviral vectors is safe, and mediates stable gene marking in macaque peripheral blood following a single transplant using myeloablative conditioning and without methods of selection for gene-modified cells. Applicants have also shown that animals transplanted with Cal-1 transduced CD34+ cells exhibit long-term, multi-lineage engraftment of gene-marked cells, and that these cells are capable of resisting SHIV challenge ex vivo and in vivo. Indeed, ex vivo assays show that Cal-1 transduction efficiency of CD34+ cells correlates with gene marking levels in peripheral blood and tissues of transplanted animals measured prior to infection, that CD4+ cells from Cal-1-transplanted animals are resistant to ex vivo SHIV infection, and that gene-marked cells undergo virus-dependent positive selection. Applicants have also shown in subset sorting experiments that there exists a preferential increase in marking in SHIV-susceptible subsets, namely in the T-cell compartment.

Based on at least these findings, Applicants believe that the incorporation of one or more transcriptional gene silencing elements into the existing Cal-1 vector will yield a triple or quadruple construct vector that is at least as safe and at least as efficacious as Cal-1 (or at least as safe and efficacious as a vector comprising one or more transcriptional gene silencing elements). As such, the combined potency and toxicity of the triple and quadruple combination lentiviral (LV) constructs (see FIG. 10) will be tested in a series of in vitro and in vivo experiments, with the specific aim of demonstrating whether combining the effect of the expression vector comprising shRNA against CCR5 and/or the C46 fusion inhibitor) with the effect of shRNAs inducing transcriptional gene silencing (TGS) from the same lentiviral vector is complementary or even synergistic in terms of control of the viral reservoir. There is now increasing evidence that such multiplexed constructs can efficiently express and allow processing of multiple shRNAs using combinations of H1, U6 and U1 promoters.

2a) Once the lentiviral constructs of the present disclosure are synthesized, we will demonstrate that there is effective and expected levels of expression of each of the TGS inducing and CCR5 inhibiting shRNAs and that they are as effectively expressed and processed.

2b) Applicants will demonstrate lentiviral constructs of the present disclosure can induce TGS as efficiently as the transcriptional gene silencing element alone by monitoring the dynamics of infection with a CXCR4 tropic virus (NL4.3) and a VZV-G pseudotyped virus. Both these viruses will evade the CCR5 shRNA and the latter will evade C46, allowing the degree of silencing achieved via shRNA-induced TGS expressed from constructs of the present disclosure to be assessed in vitro independent of the effects of the other nucleic acid components components (e.g. directed to CCR4 and/or C46, etc.). In parallel, using a β-lactamase (BLAM) reporter assay we will demonstrate inhibition of entry of a CCR5 using virus to demonstrate that constructs of the present disclosure have equivalent efficacy to those constructs described in U.S. Publication No. 2012/0201794. This will be confirmed by quantifying relative lentiviral marking and HIV-1 DNA in these cultures. These experiments will demonstrate the equivalence of expression and efficacy of each individual shRNA when delivered by standard (single/dual agent construct) or complex (triple/quadruple agent) lentiviral constructs.

2c) Once this has been demonstrated, experiments similar to those described in PCT/AU2015/050507 will be conducted to compare the efficacy and extent of off-target effects between (1) the expression vectors/constructions as described in U.S. Publication No. 2012/0201794, (2) the lead TGS candidate from PCT/AU2015/050507, and (3) the constructs of the present disclosure. These experiments will demonstrate whether any of constructs of the present disclosure have greater effects than either those described in U.S. Publication No. 2012/0201794 or those described in PCT/AU2015/050507, and particularly, because they act at different stages of the life cycle, whether the effect is additive, or even synergistic.

2d) Once in vitro experiments have demonstrated at least additive effects of one of LV of the present invention, they will be utilized for in vivo assessment of efficacy in the BLT mouse model as described above in a four arm experiment comparing its efficacy directly to the lead TGS LV construct from PCT/AU2015/050507, U.S. Publication No. 2012/0201794, and empty vector. The primary end point will be the size of the integrated HIV reservoir in CD4+ T cells from the spleen. Secondary endpoints will include pVL and CD4+ T cell counts.

2e) Once the potential advantage of the LV construct of the present disclosure is demonstrated in vivo, in addition to CD34+ HSC transduction and challenge, we will reconstitute BLT mice with transduced CD4+ T cells obtained from a HIV infected donor to demonstrate that in the presence of an established virus reservoir, a lead LV construct of the present disclosure can effectively silence pre-existing integrated virus.

Outcomes: We expect that LV construct viii) will best fulfil the outcomes of a)-c) and that due to multiple virus targets the suppressive effect will be at least additive. In vivo studies are expected to confirm more potent and more prolonged control of the viral reservoir, pVL virus and maintenance of CD4+ T cells counts, both in the periphery and tissues. Preliminary toxicity data will also be obtained.

### Additional Embodiments

1. An expression vector comprising (i) at least one nucleic acid sequence encoding a transcriptional gene silencing element; and (ii) at least two other nucleic acid sequences selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry.

2. The expression vector of 1, wherein the at least one nucleic acid sequence encoding a transcriptional gene silencing element is a silencing nucleic acid which targets a sequence of a 5′ LTR of HIV.

3. The expression vector of embodiment 1, wherein the at least one nucleic acid sequence encoding a transcriptional gene silencing element is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1.

4. The expression vector of embodiment 1, wherein the at least one nucleic acid sequence encoding a transcriptional gene silencing element is selected from the group consisting of (i) a silencing nucleic acid which targets the sequence of SEQ ID NO: 1, (ii) a silencing nucleic acid which targets the sequence of SEQ ID NO: 9, (iii) a silencing nucleic acid which targets the sequence of SEQ ID NO: 17; (iv) a silencing nucleic acid which targets the sequence of SEQ ID NO: 36; (v) a silencing nucleic acid which targets a sequence having at least 95% identity with the sequence of SEQ ID NO: 1; (vi) a silencing nucleic acid which targets a sequence having at least 95% identity with the sequence of SEQ ID NO: 9; (vii) a silencing nucleic acid which targets a sequence having at least 95% identity with the sequence of SEQ ID NO: 17; and (viii) a silencing nucleic acid which targets a sequence having at least 95% identity with the sequence of SEQ ID NO: 36.

5. The expression vector of embodiment 1, wherein the at least one nucleic acid sequence encoding a transcriptional gene silencing element is an RNA duplex comprising a sense strand and an antisense strand, wherein the sense strand comprises a sequence having at least 95% identity to one of the sequence of SEQ ID NO: 6, the sequence of SEQ ID NO: 14, the sequence of SEQ ID NO: 22, or the sequence of SEQ ID NO: 40.

6. The expression vector of embodiment 1, wherein the at least one nucleic acid sequence encoding a transcriptional gene silencing element is an siRNA selected from the group consisting of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; (iii) an siRNA comprising a sense strand having the sequence of SEQ ID NO: 22, and an antisense strand having the sequence of SEQ ID NO: 23; and (iv) an siRNA comprising a sense strand having the sequence of SEQ ID NO: 40, and an antisense strand having the sequence of SEQ ID NO: 41.

7. The expression vector of embodiment 1, wherein the at least one nucleic acid sequence encoding a transcriptional gene silencing element is an shRNA selected from the group consisting of (i) an shRNA having the sequence of SEQ ID NO: 8; (ii) an shRNA having the sequence of SEQ ID NO: 16; (iii) an shRNA having the sequence of SEQ ID NO: 24; and (iv) an shRNA having the sequence of SEQ ID NO: 42.

8. The expression vector of embodiment 1, wherein the nucleic acid sequence that encodes a nucleic acid molecule that inhibits an HIV co-receptor is a siRNA or shRNA having a double-stranded region, wherein a first portion of the double-stranded region comprises a sequence that is identical to part of a sequence of the HIV co-receptor, and wherein a second portion of the double-stranded region comprises a sequence that is complementary to another part of the sequence of the HIV co-receptor.

9. The expression vector of embodiment 8, wherein the HIV co-receptor is CCR5 or CXCR4.

10. The expression vector of embodiment 8, wherein the shRNA has a sequence of SEQ ID NO: 25.

11. The expression vector of embodiment 8, wherein the inhibitor of the HIV co-receptor is capable of reducing expression of the HIV co-receptor when the vector is expressed in a host cell.

12. The expression vector of embodiment 1, wherein the HIV fusion inhibitor protein is a C46 protein.

13. The expression vector of embodiment 1, wherein the HIV fusion inhibitor protein has the sequence of SEQ ID NO: 26.

14. The expression vector of embodiment 1, wherein the protein that inhibits HIV replication is selected from the group consisting of human TRIM5α, rhesus TRIM5α, chimeric TRIM5α, a human TRIMS-cyclophilin fusion protein, cyclophilin, E3 ubiquitin, APOBEC3G, and bone marrow stromal cell antigen 2 (BST-2).

15. The expression vector of embodiment 1, wherein the at least two other nucleic acid sequences comprise (a) a nucleic acid sequence that encodes a nucleic acid molecule that inhibits an HIV co-receptor; and (b) one of a nucleic acid sequence that encodes an HIV fusion inhibitor protein or a nucleic acid sequence encoding an inhibitor of HIV replication.

16. The expression vector of embodiment 1, wherein the at least two other nucleic acid sequences comprise a nucleic acid sequence that encodes a nucleic acid molecule that inhibits an HIV co-receptor; and a nucleic acid sequence that encodes an HIV fusion inhibitor protein.

17. The expression vector of embodiment 1, wherein the at least two other nucleic acid sequences comprise a nucleic acid sequence that encodes a nucleic acid molecule that inhibits an HIV co-receptor; and a nucleic acid sequence encoding an inhibitor of HIV replication.

18. The expression vector of embodiment 1, wherein the expression vector comprises at least two nucleic acids encoding a transcriptional gene silencing element.

19. The expression vector of embodiment 1, wherein the expression vector comprises three nucleic acids encoding a transcriptional gene silencing element.

20. The expression vector of embodiment 1, wherein the expression vector is a viral vector.

21. The expression vector of embodiment 20, wherein the viral vector is a lentiviral vector or a retroviral vector.

22. The expression vector of embodiment 21, wherein the lentiviral vector is self-inactivating.

23. The expression vector of embodiment 1, wherein the expression vector, when expressed in a host cell, confers resistance to infection by (1) X4- and R5-tropic HIV strains, (2) highly active antiretroviral therapy (HAART) resistant HIV strains or (3) X4- and R5-tropic HAART-resistant HIV strains.

24. The expression vector of embodiment 1, wherein each of the nucleic acids are expressed by separate promoters.

25. The expression vector of embodiment 1, wherein at least two of the nucleic acids are expressed by a same promoter.

26. An expression vector comprising a first nucleic acid sequence encoding an inhibitory nucleic acid capable of reducing expression of an HIV co-receptor; a second nucleic acid sequence encoding an HIV fusion inhibitor protein; and a third nucleic acid sequence encoding a silencing nucleic acid that targets a sequence with a 5′ LTR region of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1.

27. The expression vector of embodiment 26, wherein, the first nucleic acid sequence is operably linked to a first promoter; the second nucleic acid sequence is operably linked to a second promoter; and the third nucleic acid sequence is operably linked to a third promoter.

28. The expression vector of embodiment 27, wherein at least two of the first, second, and third promoters are the same.

29. The expression vector of embodiment 27, wherein the first, second, and third promoters are different.

30. The expression vector of embodiment 26, further comprising a fourth nucleic acid, wherein the fourth nucleic acid is selected from the group consisting of another nucleic acid that targets a sequence of the 5′ LTR of HIV, a nucleic acid sequence encoding an inhibitor of HIV replication; and a nucleic acid sequence encoding an inhibitor of viral entry; wherein the fourth nucleic acid is operably linked to a fourth promoter.

31. The expression vector of embodiment 26, wherein the expression vector comprises at least two silencing nucleic acids that target sequences within the 5′LTR of HIV.

32. A host cell comprising the expression vector as in any of embodiments 1 to 31.

33. The host cell of embodiment 32, wherein the host cell is a hematopoietic progenitor/stem cell, a monocyte, a macrophage, a peripheral blood mononuclear cell, a CD4+ T lymphocyte, a CD8+ T lymphocyte, or a dendritic cell.

34. A composition comprising any of the expression vectors as in any of embodiments 1 to 31.

35. The composition of embodiment 34, further comprising a pharmaceutically acceptable carrier.

36. The composition of embodiment 34, wherein the composition is formulated as an emulsion.

37. The composition of embodiment 34, wherein the composition is formulated with micelles or nanoparticles.

38. The composition of embodiment 34, wherein the compositions are encapsulated within a polymer.

39. The composition of embodiment 34, wherein the compositions are encapsulated within liposomes.

40. The composition of embodiment 34, wherein the compositions are encapsulated within minicells, nanoparticles, or nanocapsules.

41. A method of inhibiting HIV gene transcription or replication in a cell infected with HIV, comprising contacting the cell with an effective amount of the expression vector as in any of embodiments 1 to 31 or a composition as in any of embodiments 34 to 40.

42. A method of treating HIV infection in a subject comprising administering to the subject an effective amount of the expression vector as in any of embodiments 1 to 31 or a composition as in any of embodiments 34 to 40.

43. A method of preventing or reducing HIV infection in a subject comprising administering to the subject an effective amount of the expression vector as in any of embodiments 1 to 31 or a composition as in any of embodiments 34 to 40.

44. A method of preventing or reducing a productive HIV infection in a subject not suffering from an HIV infection, comprising administering to the subject an effective amount of the expression vector as in any of embodiments 1 to 31 or a composition as in any of embodiments 34 to 40.

45. A composition comprising (i) an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) a nucleic acid that encodes at least one transcriptional gene silencing element which targets a sequence within the 5′ LTR of HIV.

46. The composition of embodiment 45, wherein the expression vector is a lentiviral vector and comprises a nucleic acid sequence that encodes a nucleic acid molecule that inhibits an HIV co-receptor, and a nucleic acid sequence that encodes an HIV fusion inhibitor protein.

47. The composition of embodiment 46, wherein the HIV co-receptor is CCRS and the HIV fusion inhibitor protein is C46.

48. The composition of embodiment 45, wherein the nucleic acid that encodes at least one transcriptional gene silencing element is selected from the group consisting of (i) a silencing nucleic acid which targets the sequence of SEQ ID NO: 1, (ii) a silencing nucleic acid which targets the sequence of SEQ ID NO: 9, (iii) a silencing nucleic acid which targets the sequence of SEQ ID NO: 17; (iv) a silencing nucleic acid which targets the sequence of SEQ ID NO: 36; (v) a silencing nucleic acid which targets a sequence having at least 95% identity with the sequence of SEQ ID NO: 1; (vi) a silencing nucleic acid which targets a sequence having at least 95% identity with the sequence of SEQ ID NO: 9; (vii) a silencing nucleic acid which targets a sequence having at least 95% identity with the sequence of SEQ ID NO: 17; and (viii) a silencing nucleic acid which targets a sequence having at least 95% identity with the sequence of SEQ ID NO: 36.

49. The composition of embodiment 45, wherein the nucleic acid that encodes at least one transcriptional gene silencing element is an RNA duplex comprising a sense strand and an antisense strand, wherein the sense strand comprises a sequence having at least 95% identity to one of the sequence of SEQ ID NO: 6, the sequence of SEQ ID NO: 14, the sequence of SEQ ID NO: 22, or the sequence of SEQ ID NO: 40.

50. The composition of embodiment 45, wherein the nucleic acid that encodes at least one transcriptional gene silencing element is an siRNA selected from the group consisting of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; (iii) an siRNA comprising a sense strand having the sequence of SEQ ID NO: 22, and an antisense strand having the sequence of SEQ ID NO: 23; and (iv) an siRNA comprising a sense strand having the sequence of SEQ ID NO: 40, and an antisense strand having the sequence of SEQ ID NO: 41.

51. The composition of embodiment 45, wherein the nucleic acid that encodes at least one transcriptional gene silencing element is an shRNA selected from the group consisting of (i) an shRNA having the sequence of SEQ ID NO: 8; (ii) an shRNA having the sequence of SEQ ID NO: 16; (iii) an shRNA having the sequence of SEQ ID NO: 24; and (iv) an shRNA having the sequence of SEQ ID NO: 42.

52. The composition of embodiment 45, further comprising a pharmaceutically acceptable carrier.

53. A method of inhibiting HIV gene transcription or replication in a cell infected with HIV, comprising contacting the cell with an effective amount of the composition as in any of embodiments 45 to 52.

54. A method of treating HIV infection in a subject, comprising administering to the subject an effective amount of the composition as in any of embodiments 45 to 52.

55. A method of preventing or reducing HIV infection in a subject comprising administering to the subject an effective amount of the composition as in any of embodiments 45 to 52.

56. A method of preventing or reducing a productive HIV infection in a subject not suffering from an HIV infection comprising administering to the subject an effective amount of the composition as in any of embodiments 45 to 52.

57. A method of treating HIV infection in a subject comprising co-administering (i) an effective amount of an expression vector comprising at least two nucleic acids, wherein the at least two nucleic acids are selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry; and (ii) an effective amount of at least one silencing nucleic acid selected from the group consisting of (a) a silencing nucleic acid which targets the sequence of SEQ ID NO: 1, (b) a silencing nucleic acid which targets the sequence of SEQ ID NO: 9, (c) a silencing nucleic acid which targets the sequence of SEQ ID NO: 17; (d) a silencing nucleic acid which targets the sequence of SEQ ID NO: 36; (e) a silencing nucleic acid which targets a sequence having at least 95% identity with the sequence of SEQ ID NO: 1; (f) a silencing nucleic acid which targets a sequence having at least 95% identity with the sequence of SEQ ID NO: 9; (g) a silencing nucleic acid which targets a sequence having at least 95% identity with the sequence of SEQ ID NO: 17; and (h) a silencing nucleic acid which targets a sequence having at least 95% identity with the sequence of SEQ ID NO: 36.

58. The method of embodiment 57, wherein the co-administering is simultaneous.

59. The method of embodiment 57, wherein the expression vector and the at least one transcriptional gene silencing element are administered at different times.

60. The method of embodiment 57, wherein the expression vector comprises a nucleic acid sequence that encodes a nucleic acid molecule that inhibits an HIV co-receptor; and a nucleic acid sequence that encodes an HIV fusion inhibitor protein.

61. The method of embodiment 60, wherein the HIV co-receptor is CCR5 and the HIV fusion inhibitor protein is C46.

62. A method of preventing or reducing HIV infection in a subject comprising co-administering (i) an effective amount of an expression vector comprising at least two nucleic acids, wherein the at least two nucleic acids are selected from the group consisting of a nucleic acid sequence that encodes an inhibitor of an HIV co-receptor; a nucleic acid sequence that encodes a protein that inhibits HIV fusion to a target cell; a nucleic acid sequence that encodes a protein that inhibits HIV replication; and a nucleic acid sequence that encodes an inhibitor of viral entry; and (ii) an effective amount of at least one silencing nucleic acid selected from the group consisting of (a) a silencing nucleic acid which targets the sequence of SEQ ID NO: 1, (b) a silencing nucleic acid which targets the sequence of SEQ ID NO: 9, (c) a silencing nucleic acid which targets the sequence of SEQ ID NO: 17; (d) a silencing nucleic acid which targets the sequence of SEQ ID NO: 36; (e) a silencing nucleic acid which targets a sequence having at least 95% identity with the sequence of SEQ ID NO: 1; (f) a silencing nucleic acid which targets a sequence having at least 95% identity with the sequence of SEQ ID NO: 9; (g) a silencing nucleic acid which targets a sequence having at least 95% identity with the sequence of SEQ ID NO: 17; and (h) a silencing nucleic acid which targets a sequence having at least 95% identity with the sequence of SEQ ID NO: 36.

63. The method of embodiment 62, wherein the co-administering is simultaneous.

64. The method of embodiment 62, wherein the expression vector and the at least one transcriptional gene silencing element are administered at different times.

65. The method of embodiment 62, wherein the expression vector comprises a nucleic acid sequence that encodes a nucleic acid molecule that inhibits an HIV co-receptor; and a nucleic acid sequence that encodes an HIV fusion inhibitor protein.

66. The method of embodiment 65, wherein the HIV co-receptor is CCR5 and the HIV fusion inhibitor protein is C46.

67. A method of treating or preventing HIV infection in a subject comprising transducing hematopoietic cells with the expression vector as in any of embodiments 1 to 31 and transplanting the transduced hematopoietic cells in the subject, wherein the transduced hematopoietic cells are resistant to HIV infection.

68. Use of a composition as in any of embodiments 34 to 40 and 45-52 in the manufacture of a medicament for the treatment of a patient infected with HIV.

69. Use of a viral expression vector as in any of embodiments 1 to 31 in the manufacture of a medicament for the treatment of a patient infected with HIV.

70. An expression vector comprising a first nucleic acid encoding an shRNA having a sequence of SEQ ID NO: 25; a second nucleic acid sequence encoding for a C46 protein; and a third nucleic acid sequence encoding a silencing nucleic acid that targets one of a sequence of a 5′ LTR of HIV selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1; wherein the first nucleic acid sequence is operably linked to an H1 pol III promoter, the second nucleic acid sequence is operably linked to a UbiquitinC pol II promoter, the third nucleic acid sequence operably linked to a U6 promoter.

71. The expression vector of embodiment 70, wherein the second nucleic acid sequence encoding for the C46 protein has the sequence of SEQ ID NO: 26.

72. The expression vector of any of embodiments 70 to 71, wherein the third nucleic acid sequence encoding a transcriptional gene silencing element is an siRNA selected from the group consisting of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; (iii) an siRNA comprising a sense strand having the sequence of SEQ ID NO: 22, and an antisense strand having the sequence of SEQ ID NO: 23; (iv) an siRNA comprising a sense strand having the sequence of SEQ ID NO: 40, and an antisense strand having the sequence of SEQ ID NO: 41.

73. The expression vector of any of embodiments 70 to 71, wherein the third nucleic acid sequence encoding a transcriptional gene silencing element is an shRNA selected from the group consisting of (i) an shRNA having the sequence of SEQ ID NO: 8; (ii) an shRNA having the sequence of SEQ ID NO: 16; (iii) an shRNA having the sequence of SEQ ID NO: 24; and (iv) an shRNA having the sequence of SEQ ID NO: 42.

74. The expression vector of any of embodiments 70 to 71, further comprising a fourth nucleic acid sequence, wherein the fourth nucleic acid sequence is another of a sequence of a 5′ LTR of HIV selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1; and wherein the fourth nucleic acid sequence is operably linked to a U1 promoter.

75. The expression vector of embodiment 74, wherein the fourth nucleic acid sequence encoding a transcriptional gene silencing element is an siRNA selected from the group consisting of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; (iii) an siRNA comprising a sense strand having the sequence of SEQ ID NO: 22, and an antisense strand having the sequence of SEQ ID NO: 23; (iv) an siRNA comprising a sense strand having the sequence of SEQ ID NO: 40, and an antisense strand having the sequence of SEQ ID NO: 41.

76. The expression vector of embodiment 74, wherein the fourth nucleic acid sequence encoding a transcriptional gene silencing element is an shRNA selected from the group consisting of (i) an shRNA having the sequence of SEQ ID NO: 8; (ii) an shRNA having the sequence of SEQ ID NO: 16; (iii) an shRNA having the sequence of SEQ ID NO: 24; and (iv) an shRNA having the sequence of SEQ ID NO: 42.

77. An expression vector comprising comprising a first nucleic acid sequence encoding an inhibitory nucleic acid capable of reducing expression of an HIV co-receptor; a second nucleic acid sequence encoding an HIV fusion inhibitor protein, wherein the second nucleic acid is not T20; and a third nucleic acid sequence encoding a silencing nucleic acid that targets a sequence with a 5′ LTR region of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1.

78. The expression vector of embodiment 77, wherein the second nucleic acid sequence encoding an HIV fusion inhibitor protein has the sequence of SEQ ID NO: 26.

79. A method of treating HIV infection in a subject, comprising administering to the subject an effective amount of the composition as in any of embodiments 70 to 78.

80. A method for treating a human host patient infected with HIV, comprising extracting hematopoietic stem cells from circulating blood or bone marrow of the host patient; treating the hematopoietic stem cells with the expression vector as in any of embodiments 1 to 31; and administering or transplanting the treated hematopoietic stem cells into the same host patient.

### STATEMENT OF INDUSTRIAL APPLICABILITY

The present disclosure has applicability in the field of medicine, e.g. gene therapy.

All publications mentioned in this specification are herein incorporated by reference in their entirety. It will be appreciated by persons skilled in the art that numerous variations and/or modifications may be made to the disclosure as shown in the specific embodiments without departing from the spirit or scope of the disclosure as broadly described. The present embodiments are, therefore, to be considered in all respects as illustrative and not restrictive.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended embodiments.

1. An expression vector comprising (i) at least one nucleic acid sequence encoding a transcriptional gene silencing element; and (ii) at least two other nucleic acid sequences selected from the group consisting of a nucleic acid sequence that encodes a nucleic acid molecule that inhibits an HIV co-receptor; a nucleic acid sequence that encodes an HIV fusion inhibitor protein; a nucleic acid sequence encoding an inhibitor of HIV replication; and a nucleic acid sequence encoding an inhibitor of viral entry.

2. The expression vector of claim 1, wherein the at least one nucleic acid sequence encoding the transcriptional gene silencing element is selected from the group consisting of (i) a silencing nucleic acid which targets a sequence from about position 143 to about position 161 of a 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1.

3. The expression vector of claim 1, wherein the at least one nucleic acid sequence encoding the transcriptional gene silencing element is selected from the group consisting of (i) a silencing nucleic acid which targets the sequence of SEQ ID NO: 1, (ii) a silencing nucleic acid which targets the sequence of SEQ ID NO: 9, (iii) a silencing nucleic acid which targets the sequence of SEQ ID NO: 17; (iv) a silencing nucleic acid which targets the sequence of SEQ ID NO: 36; (v) a silencing nucleic acid which targets a sequence having at least 95% identity with the sequence of SEQ ID NO: 1; (vi) a silencing nucleic acid which targets a sequence having at least 95% identity with the sequence of SEQ ID NO: 9; (vii) a silencing nucleic acid which targets a sequence having at least 95% identity with the sequence of SEQ ID NO: 17; and (viii) a silencing nucleic acid which targets a sequence having at least 95% identity with the sequence of SEQ ID NO: 36.

4. The expression vector of claim 1, wherein the at least one nucleic acid sequence encoding the transcriptional gene silencing element is an siRNA selected from the group consisting of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; (iii) an siRNA comprising a sense strand having the sequence of SEQ ID NO: 22, and an antisense strand having the sequence of SEQ ID NO: 23; and (iv) an siRNA comprising a sense strand having the sequence of SEQ ID NO: 40, and an antisense strand having the sequence of SEQ ID NO: 41.

5. The expression vector of claim 1, wherein the at least one nucleic acid sequence encoding the transcriptional gene silencing element is an shRNA selected from the group consisting of (i) an shRNA having the sequence of SEQ ID NO: 8; (ii) an shRNA having the sequence of SEQ ID NO: 16; (iii) an shRNA having the sequence of SEQ ID NO: 24; and (iv) an shRNA having the sequence of SEQ ID NO: 42.

6. The expression vector of claim 1, wherein the protein that inhibits HIV replication is selected from the group consisting of human TRIM5α, rhesus TRIM5α, chimeric TRIM5α, a human TRIMS-cyclophilin fusion protein, cyclophilin, E3 ubiquitin, APOBEC3G, and bone marrow stromal cell antigen 2 (BST-2).

7. The expression vector of claim 1, wherein the at least two other nucleic acid sequences comprise a nucleic acid sequence that encodes a nucleic acid molecule that inhibits an HIV co-receptor and a nucleic acid sequence that encodes an HIV fusion inhibitor protein.

8. The expression vector of claim 7, wherein the nucleic acid sequence that encodes the nucleic acid molecule that inhibits an HIV co-receptor has a sequence of SEQ ID NO: 25.

9. The expression vector of claim 7, wherein the nucleic acid sequence that encodes the HIV fusion inhibitor protein has a sequence of SEQ ID NO: 26.

10. The expression vector of claim 1, wherein the expression vector comprises at least two nucleic acids encoding transcriptional gene silencing elements.

11. The expression vector of claim 1, wherein the viral vector is a self-inactivating lentiviral vector or a retroviral vector.

12. The expression vector of claim 1, wherein each of the nucleic acids are expressed by separate promoters.

13. A method for treating a human host patient infected with HIV, comprising extracting hematopoietic stem cells from circulating blood or bone marrow of the host patient; treating the hematopoietic stem cells with the expression vector of claim 1; and administering or transplanting the treated hematopoietic stem cells into the same host patient.

14. A host cell comprising the expression vector of claim 1.

15. A composition comprising the expression vector of claim 1 and a pharmaceutically acceptable carrier or excipient.

16. The composition of claim 15, wherein the composition is encapsulated within minicells, nanoparticles, or nanocapsules.

17. An expression vector comprising a first nucleic acid sequence encoding an inhibitory nucleic acid capable of reducing expression of an HIV co-receptor; a second nucleic acid sequence encoding an HIV fusion inhibitor protein, wherein the second nucleic acid does not encode T20; and a third nucleic acid sequence encoding a silencing nucleic acid that targets a sequence within a 5′ LTR region of HIV, wherein the third nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1.

18. The expression vector of claim 17, wherein the third nucleic acid sequence encoding the transcriptional gene silencing element is an shRNA selected from the group consisting of (i) an shRNA having the sequence of SEQ ID NO: 8; (ii) an shRNA having the sequence of SEQ ID NO: 16; (iii) an shRNA having the sequence of SEQ ID NO: 24; and (iv) an shRNA having the sequence of SEQ ID NO: 42.

19. The expression vector of claim 17, further comprising a fourth nucleic acid sequence encoding another silencing nucleic acid that targets a sequence within the 5′ LTR region of HIV, wherein the fourth nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, (iii) a silencing nucleic acid which targets a sequence from about position 205 to about position 223 of the 5′ LTR of HIV-1; and (iv) a silencing nucleic acid which targets a sequence from about position 350 to about position 368 of the 5′ LTR of HIV-1; and wherein the fourth nucleic acid sequence is different than the third nucleic acid sequence.

20. The expression vector of claim 17, further comprising a fourth nucleic acid sequence encoding an shRNA selected from the group consisting of (i) an shRNA having the sequence of SEQ ID NO: 8; (ii) an shRNA having the sequence of SEQ ID NO: 16; (iii) an shRNA having the sequence of SEQ ID NO: 24; and (iv) an shRNA having the sequence of SEQ ID NO: 42.
